(19) United States
(12) Patent Application Publication
(10) Pub. No.: US 2023/0381246 A1
(43) Pub. Date: Nov. 30, 2023

(54) GENETICALLY DIVERSE AKKERMANSIA STRAINS AND METHODS OF USING SAME

(71) Applicant: DUKE UNIVERSITY, Durham, NC (US)

(72) Inventors: Raphael VALDIVIA, Durham, NC (US); Bradford BECKEN, Durham, NC (US); Lauren DAVEY, Durham, NC (US)

(21) Appl. No.: 18/032,781

(22) PCT Filed: Oct. 19, 2021
(86) PCT No.: PCT/US2021/055623

§ 371 Date: Apr. 19, 2023

Related U.S. Application Data
(60) Provisional application No. 63/167,973, filed on Mar. 30, 2021.
(60) Provisional application No. 63/093,589, filed on Oct. 19, 2020.

(51) Int. Cl. A61K 35/741; A61P 3/04

(52) U.S. Cl. CPC A61K 35/741; A61P 3/04; A61K 2035/115

(57) ABSTRACT

The present invention provides compositions and methods using one or more strains of Akkermansia muciniphila. Particularly, the compositions comprising one or more A. muciniphila strains may be used to add in reduction in obesity and in methods of recolonizing the flora of the gut of a subject, as described in the disclosure.

### CROSS-REFERENCE TO RELATED APPLICATIONS

This application claims priority to U.S. Provisional Application No. 63/093,589 filed on Oct. 19, 2020 and U.S. Provisional Application No. 63/167,973 filed on Mar. 30, 2021, the contents of which are incorporated by reference in their entireties.

### STATEMENT REGARDING FEDERALLY SPONSORED RESEARCH

This invention was made with government support under grant number AI142376 and CA249243 awarded by the National Institutes of Health. The government has certain rights in this invention.

### SEQUENCE LISTING

A Sequence Listing accompanies this application and is submitted as an ASCII text file of the sequence listing named “155554_00626_ST25.txt” which is 3,394 bytes in size and was created on Oct. 19, 2021. The sequence listing is electronically submitted via EFS-Web with the application and is incorporated herein by reference in its entirety.

### BACKGROUND

The gastrointestinal (GI) microbiota comprises a complex community of bacteria, fungi, and archaea that significantly influence the metabolic and immunological health of their human and animal hosts (recently reviewed in (1)). The global obesity epidemic has focused attention on the role that the microbiota plays in regulating energy acquisition and inflammation and how these activities impact the development of metabolic disease and diabetes (2). Western style diets, in particular, lead to microbiotas of lower taxonomical diversity and metabolic capacity which in turn enhance the risk for developing inflammatory disorders like diabetes, obesity and cardiovascular disease (3-5).

Akkermansia muciniphila is a Gram negative anaerobic bacterium of the phylum Verrucomicrobia that can use GI mucins as a sole carbon and nitrogen source (6, 7). A. muciniphila has attracted considerable attention because an increased abundance of Akkermansia in the GI tract correlates with many positive human health outcomes, including protection from obesity, diabetes and metabolic disease (8-11). Indeed, lean individuals show an increased representation of Verrucomicrobia in their fecal microbiomes as assessed by 16S rRNA gene profiling (9). A. muciniphila is prevalent in the colon and has been reported to comprise between 1-4% of the total bacteria in healthy adult fecal samples (12). In mice, repeated administration of A. muciniphila ameliorates the impact of high fat diets in inducing obesity and strengthens the function of the GI epithelial barrier though the activation of Toll-like Receptor 2 (TLR2) (13, 14). In proof-of-concept trials in humans, administration of live or pasteurized A. muciniphila was sufficient to improve insulin sensitivity and reduced insulinemia (15).

Two recent pangenomic studies of Akkermansia, including a comparison of thirty five A. muciniphila genomes reconstructed from metagenomic sequences (16) and thirty nine A. muciniphila isolated strains (17), led to the identification of four distinct phylogroups (clades AmI-IV). An analysis of the genomes of representative members of these phylogroups revealed phylogroup-specific functions such as the ability of AmII strains to synthesize corrin rings (16) and thus potentially outcompete other phylogroups when levels of vitamin B12 precursors in the GI tract are scarce. Given that multiple A. muciniphila phylogroups are found in humans, we asked what is the genomic and phenotypic diversity of A. muciniphila isolated from children with the long term goal of determining if there are correlates between strain and phylogroup abundance and specific health outcomes. To begin to address these questions, we used fecal samples collected by the Pediatric Obesity Microbiome and Metabolome Study (POMMS) (18) to isolate 71 unique strains of A. muciniphila. Participants included adolescents with healthy weight at a single timepoint only, and adolescents with obesity at baseline and then at 6-months following a behavioral lifestyle intervention. A sub-sample of the participants with obesity additionally received pharmacotherapy or bariatric surgery during the study period. We performed a detailed genotypic and phenotypic analysis of these isolates and determined that POMMS A. muciniphila strains belonged to three main phylogroups/clades. Several in vitro traits, like growth rates in mucin, resistance to ambient oxygen, self-aggregation and binding to epithelial surfaces were linked to specific phylogroups. Furthermore, we determined that even though specific phylogroups displayed GI tract colonization advantages in antibiotic treated mice, the parameters influencing Akkermansia colonization in humans are more complex. Accordingly, there is a need for compositions and methods that can be useful for altering the gut bacteria flora and that can be used in the treatment of various human pathologies.

### SUMMARY

In a first aspect, the present invention provides compositions comprising at least one strain of Akkermansia muciniphila in Table S3 and a pharmaceutically acceptable carrier.

In a second aspect, the present invention provides methods for the manufacture of a product. The methods comprise: (a) providing a strain of Akkermansia muciniphila selected from Table S3, and (b) adding the strain to an edible composition, thereby obtaining the product.

In a third aspect, the present invention provides methods of recolonizing the gut of a subject. The methods comprise administering a composition described herein in an amount capable of recolonizing the gut. Recolonization may benefit a subject whose gut microbiome is out of balance or has been depleted by an antibiotic regimen, for example.

In a fourth aspect, the present invention provides methods of altering the microbial composition of the gut of a subject. The methods comprise administering a composition described herein in an amount effective to alter the microbial composition within the subject.

In a fifth aspect, the present invention provides methods of treating or preventing a disease or condition in a subject. The methods comprise administering to the subject a therapeutically effective amount of a strain of Akkermansia muciniphila described herein such that the disease condition is treated.

In a sixth aspect, the present invention provides methods of reducing obesity in a subject in need thereof. The methods comprise administering to the subject a therapeutically effective amount of a composition described herein to reduce obesity.

In a seventh aspect, the present invention provides methods of enhancing the response to a cancer immunotherapy in a subject. The methods comprise administering to the subject a therapeutically effective amount of a composition described herein.

In an eighth aspect, the present invention provides method of promoting healthy aging, alleviate neurobehavioral and neurodegenerative disorders, promote healthy mucosal and systemic immunity. The methods comprise administering to the subject a therapeutically effective amount of a composition described herein.

### BRIEF DESCRIPTION OF THE DRAWINGS

FIG. 1. Relative abundance of Verrucomicrobia in stool samples of children with obesity. Phylum level assessment of the composition of bacterial communities in fecal samples derived from children with obesity before and 6 months after undergoing treatment for weight loss (A), and representative children of healthy weight (B) enrolled in the same study. The identity and relative representation of bacteria within each stool sample was determined by amplification and sequencing of the 16S RNA locus. The normalized fraction (%) of Verrucomicrobia is provided under the Visit (V) number. ND: not detected.

FIG. 2. A. muciniphila human isolates display variance in phenotypes relevant to gastrointestinal colonization. (A) A. muciniphila strains display distinct growth rates in porcine gastric mucin. The growth rates of A. muciniphila isolates were monitored in liquid media over a period of 96 h. Representative examples of fast and slow growers are shown in relation to the typed A. muciniphila strain MucT. (B) A. muciniphila strains agglutinate. Selected strains shown as examples of rapid sedimentation of A. muciniphilagrown in mucin medium. (C) A. muciniphila vary in their ability to attach to epithelial surfaces. Adhesion of selected A. muciniphila strains to HT29 colonic epithelial cells was assessed by immunofluorescence microscopy. HT29 nuclei were detected with Hoechst (blue) and bacteria with polyclonal anti-Akkermansia antiserum (white-left panels; green-right panels). Scale bar 100 mm. (D) A. muciniphilastains vary in their tolerance to ambient oxygen. Strains grown in BHI supplemented with mucin were exposed to ambient oxygen for 0, 12, or 24 hours on BHI-mucin agar plates followed by outgrowth under anaerobic conditions. Oxygen sensitivity was assessed by enumerating CFUs on agar plates.

FIG. 3. Activation of TLR2 and TLR4 by A. muciniphila isolates. (A) TLR2 and TLR4 are the main microbial sensors required for the recognition of A. muciniphila. Bone marrow derived macrophages from the selected tlr knockout mice were incubated with A. muciniphila MucT at a ratio 5 (white bars) or 0.5 (grey bars) per BMDM for 6 h followed by measuring TNF□ or IL-6 as readouts of activation. BMDM activation only occurs in cells expressing either Tlr4 or Tlr2, with Tlr4 displaying a lower threshold for A. muciniphila stimulation. (B) A. muciniphilastrains vary in their ability to activate TLR2 and TLR4. Selected strains grown on mucin medium were incubated with HEK293 reporter cell lines expressing either TLR2 or TLR4 at ratio of 5 or 1 bacteria/cell (white and grey bars, respectively). Stimulation of TLR in these cell lines was determined by assessing the processing of a colorimetric substrate of secreted of alkaline phosphatase.

FIG. 4. A. muciniphila isolates from children belong to three different phylogroups. (A) Whole genome comparison of 40 A. muciniphila strains. The average nucleotide identity (ANI) was calculated at 96% identity using Anvi'o and PyANI. Genomes for previously published strains (16, 17) belonging to each Akkermansia phylogroup were included as controls (blue), as well as the type-strain MucT (red). Complete genomes were assembled from PacBio reads. Note that phylogroup AMI can be subdivided into two subtypes at 96% ANI threshold. (B) Circle phylogram of new A. muciniphila strains. Graph displays the pangenome of 40 sequenced isolates, three reference genomes (grey), and the type strain MucT (red). The phylogram is clustered based on gene frequency and displays gene cluster presence/absence for each genome. Selected phylogroup-specific gene groups are highlighted to show their distribution, including vitamin B12 biosynthetic gene groups, putative enterochelin transporters, Fe3+transporters, and capsule genes (labelled as pyruvyl transferases). (C) Gene set enrichment analysis of A. muciniphila phylogroups. Anvi'o was used to identify COG functions associated with specific phylogroups. The selected COGs were detected in all isolates belonging to a given phylogroup(s) and in none of the isolates belong to other phylogroups. The adjusted q-value shows significance of the enrichment between the function and the associated phylogroup corrected for multiple testing.

FIG. 5. A. muciniphila strains display phylogroup-dependent and independent phenotypes. Distribution of A. muciniphila (A) doubling times in mucin medium, (B) resistance to ambient oxygen on agar plates, (C) adherence, (D) agglutination, (E) mucin fermentation, (F) TLR2 activation, and (G) TLR4 activation. Each symbol represents a strain. AmI strains that were not subtyped into AmIa or AmIb are shown as grey dots. Details available on Table S3. P values were calculated using Kruskal-Wallis tests followed by Dunn's multiple-comparisons tests. *P<0.05, **P<0.01, ****P<0.0001.

FIG. 6. AmII and AmIV phylogroups are defective for assimilatory sulfate reduction (ASR). (A) Distribution of ASR genes among A. muciniphila phylogroups. ASR genes (top) from A. muciniphila Muc^{T} were used to search for homologs among other sequenced isolates. Blue squares indicate that a gene is present and grey squares indicate that a gene was not detected. There are two cysK homologs in Muc^{T}: Amuc_1301 and Amuc_2014. (B) Genomic context of ASR genes in Akkermansia phylogroups. The majority of the ASR genes are clustered in a single locus in the AmI phylogroup, as represented in strains Muc^{T} and Akk0500B. The AmI strain, Akk2670, lacked the entire ASR locus, although the flanking genes remained conserved in other AmI strains. The AmII strain (Akk2196) also lacked the entire ASR locus, while the AmIV strain (Akk0496) missed the locus and flanking genes. Arrows represent genes, polarity of reading frame, and the numbers above each arrow indicate the gene number in the annotated genome. The genome coordinates for each locus is shown below the arrows. (C) Addition of cysteine or NaHS enhances the growth of ASR-deficient A. muciniphila. Representative Am strains from each phylogroup were tested for growth in mucin medium with or without the addition of 1 mM L-cysteine or 40 μM NaHS.

FIG. 7. A. muciniphila phylogroups AmII and AmIV strains outcompete AmI in antibiotic pre-treated mice. Mice were either untreated (A and C) or treated (B and D) with antibiotics (Ab) and gavaged with a three-phylogroup strain mix containing an equal amount of phylogroups AmIa (Muc^{T}), AmIb (Akk1683), and AmII (Akk0580) (A and B) or a four-phylogroup strain mix containing phylogroups AmIa (Muc^{T}), AmIb (Akk1570), AmII (Akk0580), and AmIV (Akk0490) (C and D). The AmIa strain identified in mice that had not been pre-treated with Ab (A and C) represents the endogenous mouse Akkermansia. Each point represents the average of three cages (n=4 mice/cage for AB and n=2mice/cage for CD) and error bars represent the standard deviation. The Amla strain in A-C represent the endogenous mouse A. muciniphila strain found at the Duke University vivarium.

FIG. 8. Evidence for single A. muciniphila phylogroup dominance in humans. A subset of stool samples from 14 patients (PID) were selected for detection and quantification of specific phylogroups by qPCR. Samples were selected for analysis if patients had provided multiple samples (n>3) throughout a 6 month period and 16S rRNA community profiling indicated the presence of Akkermansia in at least one timepoint. The size of the bubbles represents the relative abundance of Akkermansia as assessed with QIIME analysis. Triangle indicate that no Akkermansia/Verrucomicrobia 16S rRNA sequences were detected. The frequency of phylogroup types was assessed by qPCR with specific primers and are color coded. In all cases the dominant phylogroup represented >99.9% of total Akkermansia with a threshold for phylogroup identification set at a Cq value of 34 of lower (Table S5). Missing data points indicate that no sample was collected at that time point.

FIG. 9. A. muciniphila binding to HT29 colonic epithelial cell and protein-coated plated. Adherence of A. muciniphila strains isolated from patients enrolled in the POMMS to HT29 colonic epithelial cell- or BSA-coated plates. Bacteria were incubated with HT29-MTX cell- or BSA-coated plates and extensively washed, and binding was indirectly evaluated by adding synthetic medium to the wells and measuring the optical density at 600 nm after 48 to 72 h. (A) Binding is provided as the ratio of the OD600 HT29-MTX to BSA-coated plates. (B and C) The nonnormalized OD600 units for HT29-MT (B) or BSA-coated (C) plates are provided for reference. Bars represent the mean and standard deviation of 3 technical replicates from 3 independent experiments.

FIG. 10. A. muciniphila activation of HEK293-TLR reporter cell lines. (A to D) A. muciniphila strains were grown on mucin or synthetic medium and incubated with HEK293 reporter cell lines expressing either TLR1/2/6 (A and B) or TLR4 (C and D). Activation of the TLR in these cell lines was determined by assessing the secretion of alkaline phosphatase into culture supernatants. Data are reported as the normalized fold induction over the mean OD value of the entire population. Bars represent the mean and standard deviation of 3 technical replicates from 3 independent experiments.

FIG. 11. Genomic analysis of A. muciniphila strains and predicted functions. (A) Pathway enrichment analysis indicates selective acquisition and loss of metabolic pathways. To compare the metabolic capabilities of isolates, we used Anvi'o to estimate metabolism function to detect KEGG module pathways in the genomes. The lower threshold for detecting a pathway was set to 50%. Strains with 50% or less of the enzymes required for a given pathway are represented as blue squares in the heatmap. (B) Detection of selected glycoside hydrolase families by Glade. Bars represent the average number of enzymes per genome. The analysis included genomes from Glade AmIa (n=5), AmIb (n=20), AmII (n=10), and AmIV=(n=8). GH, glycoside hydrolase; NC, nonclassified glycoside hydrolases. Error bars represent the standard error of the mean (SEM).

FIG. 12. A. muciniphila phylogroup AmIV outcompetes other phylogroups in colonizing the mouse GI. (A) Fecal samples were collected prior to gavage, and DNA was extracted to assess endogenous Akkermansia levels in mice with and without antibiotic treatment. Akkermansia was detected with primers specific for the Akkermansia 16S rRNA gene (total) and with phylogroup-specific primers. (B) In vivo competition assay using a second set of representative strains for each phylogroup. A representative of each strain from a healthy control was selected as follows: AmIa (Akk2670), AmIb (Akk2650), AmII (2680), and AmIV (2750). Mice were treated with antibiotics until the endogenous Akkermansia was cleared and were subsequently gavaged with a cell suspension containing each of the phylogroups. Colonization was monitored over time by collecting fecal pellets and testing for colonization using qPCR with phylogroup-specific primers.

FIG. 13. Table S1. Summary of pediatric donors described in this study.

FIG. 14. Table S2. ASV tables and relative abundance and composition of microbial communities in POMMS samples.

FIG. 15. Tables S3. Summary of A. muciniphila isolates, phylogroups and their phenotypes.

FIG. 16. Table S4. Primers used for the identification of A. muciniphila phylogroups and quality control for specificity.

FIG. 17. Table S5. Phylogroup-specific enrichment of metabolic pathways.

FIG. 18. Table S6. Phylogroup typing in patients with obesity over a 6 month period.

### DETAILED DESCRIPTION

The present invention provides compositions comprising the strains of Akkermansia muciniphila described herein and methods of using said compositions, e.g., to alter the microbial composition of the gut and to treat disease.

The present application is based on the inventors' characterization of 71 new A. muciniphila strains that were isolated from a cohort of children and adolescents undergoing treatment for obesity (See Table S3). Based on genomic and phenotypic analysis of these strains, they found several phylogroup specific phenotypes that may affect the colonization of the GI or modulate host functions such as oxygen tolerance, adherence to epithelial cells, iron and sulfur metabolism, and exopolysaccharide biosynthesis. In antibiotic treated mice, AmIV and AmII outcompeted AmI strains. In children and adolescents, they observed high variance in A. muciniphila abundance and evidence for single phylogroup dominance, with phylogroup switching occurring in a small subset of patients. Overall, these results highlight that the ecological principles determining which A. muciniphila phylogroup predominates in humans are complex and that A. muciniphila strain genetic and phenotypic diversity may represent an important variable that should be taken into account when making inferences as to this microbe's impact on their host's health. Further, the collection of A. muciniphila strains identified by the inventors provides a foundation for the functional characterization of A. muciniphila phylogroup-specific effects on the multitude of host outcomes associated with Akkermansia colonization, including protection from obesity, diabetes, colitis, neurological diseases and enhanced response to cancer immunotherapies.

### Compositions

The present invention provides compositions comprising at least one strain of Akkermansia muciniphila in Table S3 and a pharmaceutically acceptable carrier. In Table S3 characterizes the 71 new A. muciniphila strains that were identified by the inventors. These strains represent the range of genetic and phenotypic diversity present in this species of beneficial microbe. Phenotypes of interest include, but are not limited to, diverse capsular types, ability to form biofilms (which is important for colonization), and ability to activate innate immune pathways.

“Pharmaceutically acceptable” carriers are known in the art and include, but are not limited to, suitable diluents, preservatives, solubilizers, emulsifiers, liposomes, nanoparticles, and adjuvants. Pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. Suitably, the pharmaceutical carriers are carriers that allow of the viability of the one or more A. muciniphila strains to be maintained.

The compositions of the present invention include both liquids and dried or lyophilized formulations. The compositions may include diluents of various buffer content, pH, and ionic strength, additives such as albumin or gelatin to prevent absorption to surfaces, solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances, or tonicity modifiers (e.g., lactose, mannitol). The compositions may also be incorporated into or onto preparations of polymeric compounds such as polylactic acid, polyglycolic acid, hydrogels, or onto liposomes, microemulsions, micelles, milamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

In some embodiments, the compositions comprise two or more strains of Akkermansia muciniphila in Table S3 and a pharmaceutically acceptable carrier. In some embodiments, the two or more strains are from at least two different phylogroups, as compositions comprising relatively diverse strains may provide an advantageous combination of phenotypic traits. In some embodiments, the compositions comprise three or more strains.

In some embodiments, the at least one strain of Akkermansia muciniphila is selected from the group consisting of Akk0090, Akk00915, Akk0093, Akk00945a, Akk0096, Akk01915, Akk0196, Akk0200, Akk0330, Akk0490a, Akk0496a, Akk0496b, Akk0500a, Akk0500b, Akk05415, Akk0580, Akk05815, Akk0880, Akk1370, Akk13715, Akk1376, Akk1410, Akk14115, Akk14745a, Akk14745b, Akk1476, Akk1570, Akk1573, Akk1576, Akk1610, Akk16115, Akk1613, Akk16145, Akk1616, Akk1683, Akk1700, Akk1713, Akk1750, Akk17515, Akk1756, Akk1813, Akk1820, Akk1826b, Akk1826d, Akk1863, Akk18645, Akk1866d, Akk1900, Akk1906, Akk1990, Akk2000, Akk2030, Akk2033, Akk2080, Akk2090, Akk21215, Akk2180, Akk2190, Akk2196, Akk2300, Akk2340, Akk2543, Akk2583, Akk2633, Akk2640, Akk2650, Akk2670, Akk2680, Akk2740, Akk2750, AkkB40, and combinations thereof. This is the group of A. muciniphila strains that is characterized in the Examples and found in Table S3. In another embodiment, the one or more A. muciniphila strains is from FIG. 6. In another embodiment, the one or more A. muciniphila strains are selected from Akk1610, Akk1576, Akk0880, Akk1683, Akk1756, Akk2650, Akk1370, Akk1475, Akk1990, Akk1713, Akk2670, Akk0490, Akk2750, Akk0580, Akk2680, Akk2000, Akk2190, AkkB40 and combinations thereof. Suitable combinations are understood by one skilled in the art.

The A. muciniphila strains of the present invention may provide health benefits, e.g., by preventing intestinal inflammation and enhancing gut health. Thus, in some embodiments, the compositions are formulated as a probiotic. As used herein, the term “probiotic” refers to microorganisms that are beneficial to the health of an animal host. In some embodiments, the compositions are formulated for oral administration, for example, as a food product or a food supplement

In some embodiments, the compositions formulated as a liquid, a powder, a capsule, a tablet, or a sachet for oral administration. In one embodiment, the composition is a liquid formulation. In another embodiment, the composition is a capsule or tablet in which the bacteria are freeze-dried or lyophilized such that they can be reconstituted into viable bacteria within the gut of the subject to be administered.

A capsule or tablet may include an enteric coating. In such an embodiment, an outer housing of the capsule may be made of gelatin or cellulose. Cellulose has the benefit of maintaining the formulation in intestinal fluid, disallowing premature breakdown in the upper gastrointestinal tract, so the product can reach the desired destination. Alternatively, the ingredients may be combined and formed into a tablet. In tablet form, cellulose may also be present to act as a binder to hold the tablet together. Probiotic compositions may further comprise one or more excipients to facilitate the manufacturing process by preventing the ingredients from adhering to machines. Moreover, such excipients may render the capsule or tablet form easier to swallow and digest through the intestinal tract. The excipients may be a vegetable stearate, magnesium stearate, steric acid, ascorbyl palmitate, retinyl palmitate, or hyproxypropyl methylcellulose. Additional colors, flavors, and excipients known in the art may also be added. The formulated probiotic composition may be administered as formulated (e.g., as a capsule or tablet), or may be combined with food or drink for administration.

The compositions of the present may also include lyophilized microorganisms, live cultures, or a combination thereof. In some embodiments, the compositions comprise one or more strains of A. muciniphila that is live and pasteurized.

In some embodiments, the composition of the present invention is lyophilised, pulverised and powdered. It may then be infused, dissolved such as in saline, as an enema, for local gut administration. Alternatively the powder may be encapsulated as enteric-coated capsules for oral administration. These capsules may take the form of enteric-coated microcapsules. As a powder it can preferably be provided in a palatable form for reconstitution for drinking or for reconstitution as a food additive. The composition can be provided as a powder for sale in combination with a food or drink. Typically, the food or drink is a dairy-based product or a soy-based product. In some embodiments, the composition is a food or food supplement containing a composition according to the present disclosure. In a preferred form the food or food supplement contains enteric-coated microcapsules of the composition of the invention. In a preferred form the food is yogurt.

The composition can be combined with other adjuvants such as antacids to dampen bacterial inactivation in the stomach, e.g., Mylanta, Mucaine, Gastrogel. Acid secretion in the stomach could also be pharmacologically suppressed using H2-antagonists or proton pump inhibitors, for example, but not limited to, an H2-antagonist such as ranitidine. Typically the proton pump inhibitor is omeprazole.

The composition of the invention may be in the form of an enema composition which can be reconstituted with an appropriate diluent, enteric-coated capsules, enteric-coated microcapsules, powder for reconstitution with an appropriate diluent for colonoscopic infusion, powder for reconstitution with appropriate diluent, flavoring and gastric acid suppression agent for oral ingestion, powder for reconstitution with food or drink, food or food supplement comprising enteric-coated microcapsules of the composition, powder, jelly, or liquid, among others.

### Methods

The present invention provides methods for the manufacture of a product. The methods comprise: (a) providing a strain of A. muciniphila selected from Table S3, and (b) adding the strain to an edible composition, thereby obtaining the product.

In some embodiments, the manufactured product is a probiotic food product. For example, a probiotic food product may be formulated as a milk-based product, and may be provided in milk, yogurt, cheese, or ice cream. The food product may also be formulated as a non-dairy product, such as a fruit-based product, or a soya-based product. Such foods products can be in solid or liquid/drinkable form. Further, the food product can contain all customary additives, including but not limited to, proteins, vitamins, minerals, trace elements, and other nutritional ingredients.

In another aspect, the present invention provides methods of recolonizing the gut of a subject. The methods comprise administering a composition described herein in an amount capable of recolonizing the gut. Recolonization may benefit a subject whose gut microbiome is out of balance or has been depleted by an antibiotic regimen, for example.

The present invention also provides methods of altering the microbial composition of the gut of a subject. The methods comprise administering a composition described herein in an amount effective to alter the microbial composition within the subject.

According to another embodiment of the invention there is provided a pharmaceutical composition useful for the treatment and/or prophylaxis of chronic disorders associated with the presence in the gastrointestinal tract of a mammalian host of abnormal or an abnormal distribution of microflora, which composition comprises one or more viable strains of A. muciniphila described herein (e.g., Table S3).

Further, the present invention provides methods of treating or preventing a disease or condition in a subject. The methods comprise administering to the subject a therapeutically effective amount of one or more strains of A. muciniphila described herein such that the disease condition is treated. Is some embodiments, the condition may be overweight or obesiety.

The term “effective amount” or “therapeutically effective amount” refers to an amount sufficient to effect beneficial or desirable biological and/or clinical results.

The methods of the present invention may be used to treat any disease that is influenced by imbalances in the gut microbiota. For example, such imbalances have been linked with gastrointestinal conditions such as inflammatory bowel disease (IBD) and irritable bowel syndrome (IBS), and wider systemic manifestations of disease such as obesity, type 2 diabetes, and atopy. Studies suggest that the gut microbiome may also affect diseases such as cancer, autoimmune disorders such as multiple sclerosis and autism spectrum disorder. Thus, in some embodiments, the disease or condition is selected from the group consisting of cancer, metabolic disease, inflammatory conditions, neurological diseases, and combinations thereof. In some embodiments, the disease comprises a neurological disease, such as ALS. In another embodiment, the disease comprises a metabolic disease, such as diabetes. In yet another embodiment, the disease condition comprises an inflammatory condition, such as pancreatitis. In other embodiments, the subject is overweight or obese.

In the Examples, the inventors identify and characterize A. muciniphila strains that were isolated from a cohort of children and adolescents undergoing treatment for obesity. Thus, in another aspect, the present invention provides methods of reducing obesity in a subject in need thereof. The methods comprise administering to the subject a therapeutically effective amount of a composition described herein to reduce obesity. Is some embodiments, the composition comprise one or more strains of A. muciniphila found in Table S3, particularly ones associated with healthy weight or loss of weight in a subject.

As used herein, the term “obesity” refers to a disorder involving excessive body fat that increases the risk of health problems. Obese persons are often defined as having a body mass index (BMI) of greater than 30. The body mass index (BMI) is calculated by dividing an individual's weight in kilograms by the square of their height in meters. The term “overweight” refers to excessive body fat with a BMI of from about 25-30. The term reducing obesity can be measured by a number of factors, including, for example, the reduction in the weight of the subject, the reduction in the body mass index of the individual or the reduction in the amount of body fat in the subject.

The present invention also provides methods of enhancing the response to a cancer immunotherapy in a subject. The methods comprise administering to the subject a therapeutically effective amount of a composition described herein. Enhance response to cancer therapy may be measured by methods known in the art, and include, for example, an inhibition of tumor cell growth, a decrease in tumor size, reducing in number of tumor cells, reduction in one or more symptoms associated with the cancer, or combinations thereof.

The dose of A. muciniphila required to recolonize the gut, to alter the microbial composition within the subject, or to be therapeutically effective will depend on many variables, including the current microbiome composition of the subject, as well as the subject's health status, diet, medications, physical activity, exposure to pathogens, etc. However, in some embodiments, the amount of A. muciniphila used with the methods of the present invention is between about 10³ to 10¹⁵ microorganisms per dose, preferably 1×10⁵-1×10¹⁵ microorganisms per dose (colony forming units (CFU) per dose); between about 1×10⁶-1×10¹⁴ microorganisms per dose; between about 1×10⁷-1×10¹³ microorganisms per dose; between about 1×10⁸-1×10¹² microorganisms per dose, between about 1×10⁹-1×10¹¹ microorganisms per dose; between about 1×10¹⁰-9×10¹⁰ microorganisms per dose; or about 3×10¹⁰ microorganisms per dose.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the animal is a far animal, such as cow, pig, sheep, horse, or chicken.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations were interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

The following Examples are provided by way of illustration and not by way of limitation.

### EXAMPLES

### Example 1

The mucophilic anaerobic bacterium Akkermansia muciniphila is a prominent member of the gastrointestinal (GI) microbiota and the only known species of the Verrucomicrobia phylum in the mammalian gut. A high prevalence of A. muciniphila in adult humans is associated with leanness and a lower risk for the development of obesity and diabetes. Four distinct A. muciniphila phylogenetic groups have been described, but little is known about their relative abundance in humans or how they impact human metabolic health.

In the following Example, the inventors characterized novel A. muciniphila strains that were isolated from a cohort of children and adolescents undergoing treatment for obesity. Given that multiple A. muciniphila phylogroups are found in humans, the inventors investigated the genomic and phenotypic diversity of A. muciniphila isolated from these children with the long-term goal of determining if there are correlates between strain and phylogroups abundance and specific health outcomes during weight reduction interventions in this population.

Given that multiple A. muciniphila phylogroups are found in humans, the inventors asked what is the genomic and phenotypic diversity of A. muciniphila isolated from children with the long term goal of determining if there are correlates between strain and phylogroup abundance and specific health outcomes. To begin to address these questions, we used fecal samples collected by the Pediatric Obesity Microbiome and Metabolome Study (POMMS) (18) to isolate 71 unique strains of A. muciniphila. Participants included adolescents with healthy weight at a single timepoint only, and adolescents with obesity at baseline and then at 6-months following a behavioral lifestyle intervention. A sub-sample of the participants with obesity additionally received pharmacotherapy or bariatric surgery during the study period. We performed a detailed genotypic and phenotypic analysis of these isolates and determined that POMMS A. muciniphila strains belonged to three main phylogroups/clades. Several in vitro traits, like growth rates in mucin, resistance to ambient oxygen, self-aggregation and binding to epithelial surfaces were linked to specific phylogroups. Furthermore, we determined that even though specific phylogroups displayed GI tract colonization advantages in antibiotic treated mice, the parameters influencing Akkermansia colonization in humans are more complex.

### RESULTS

### The Relative Abundance of A. muciniphila in Fecal Samples is Highly Variable in a Cohort of Children with Obesity

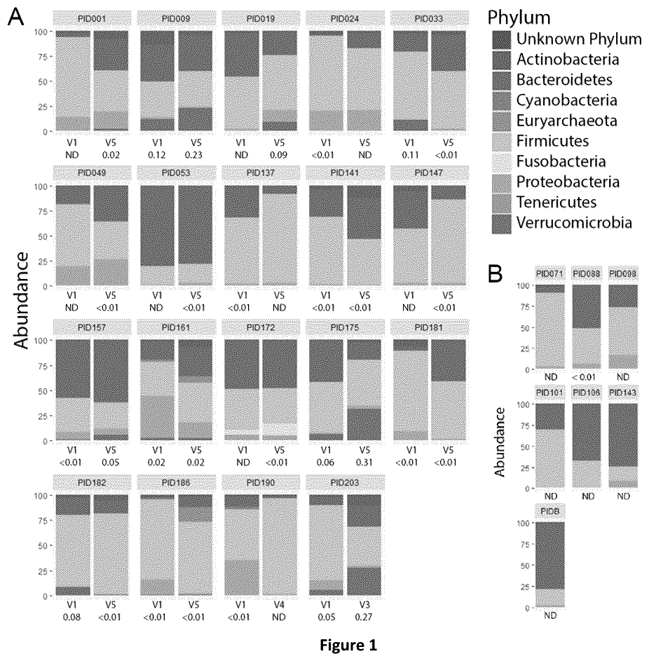

To survey the diversity of A. muciniphila strains present among healthy children and those with obesity we used fecal samples that had been collected before and after various interventions aimed at decreasing their body mass index (BMI) (18). We selected five lean controls (z-score adjusted BMI or zBMI-0.99 to 0.41) and thirty six children with extreme obesity (zBMI-1.63 to 3.18>95^{th} percentile) (Table S1). For healthy lean control children, a single stool sample was obtained at the time of enrollment. For the majority of the cohort with obesity (35/36) we used samples collected at baseline and at the end of the study (6 months). We enriched for mucolytic bacteria directly from frozen fecal material by serial passage in liquid medium with gastric porcine mucin as the sole carbon and nitrogen source, and then isolated single colonies on mucin agar plates. Bacterial colonies were purified to homogeneity and typed by sequencing the V3-4 region of the 16S rRNA locus. Overall, we cultured 71 strains of A. muciniphila from 35 children and one adult. In parallel, we performed a 16S rRNA-based survey of the bacterial communities present in selected fecal samples for which we had baseline and at least one additional visit (FIG. 1 and Table S2). A phylum-level analysis indicated that the relative abundance of Verrucomicrobia ranged from undetectable to 31% of total bacteria (Table S2).

A. muciniphila Clinical Isolates are Phenotypically Diverse

Previous analysis of microbial metagenomes indicated that there is significant diversity among A. muciniphila strains (19). To begin to address if this genetic diversity correlates with traits of relevance to the colonization and health of the human host, we performed a range of phenotypic tests that have been associated with Akkermansia biology including variations in 1) growth rates 2) the ability to form bacterial aggregates, 3) adherence to epithelial surfaces, 4) the generation of short chain fatty acids (SCFA) during mucin fermentation, 5) sensitivity to oxidative stress, and 6) activation of Toll-like receptors (TLRs).

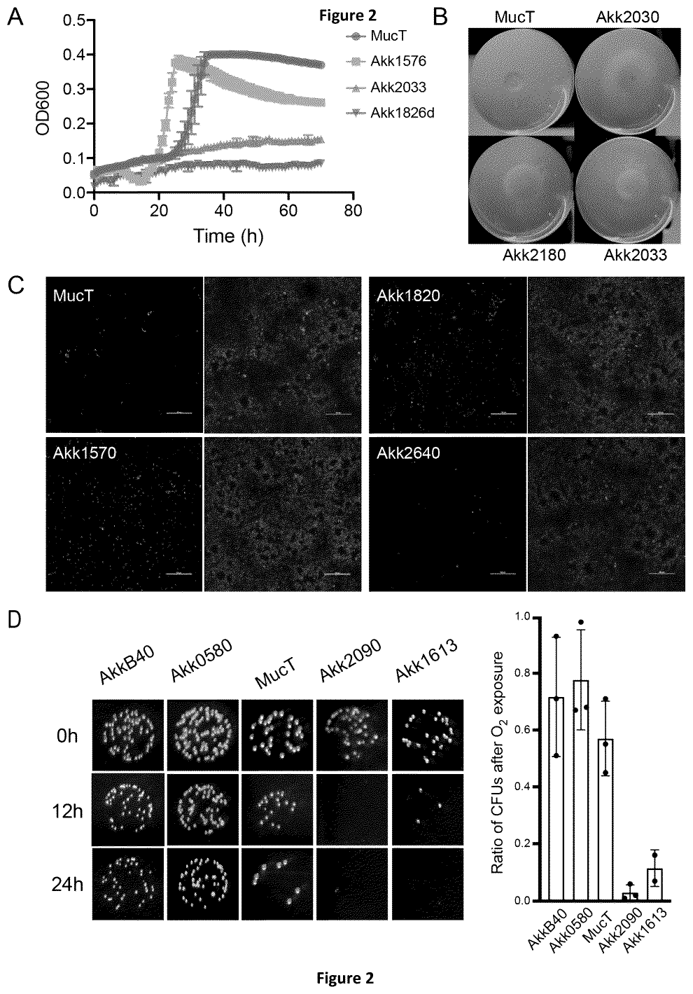

Growth rates: The growth rates among individual strains were monitored under anaerobic conditions in a semi defined synthetic medium consisting of glucose, N-acetyl glucosamine, soy peptone and threonine. A. muciniphila strains displayed doubling times ranging from 1.2-13 h. These differences were more pronounced when gastric porcine mucin was the sole carbon and nitrogen source, with doubling times ranging from 0.3 to >20 h (FIG. 2A and Table S3).

Aggregation: Some A. muciniphila isolates, unlike the reference strain A. muciniphila Muc^{T}, readily sedimented when grown in liquid culture media (FIG. 2B). We measured the extent to which strains aggregated by monitoring the changes in the optical density in samples obtained from the surface of cultures tubes that had been grown in mucin medium (Table S3).

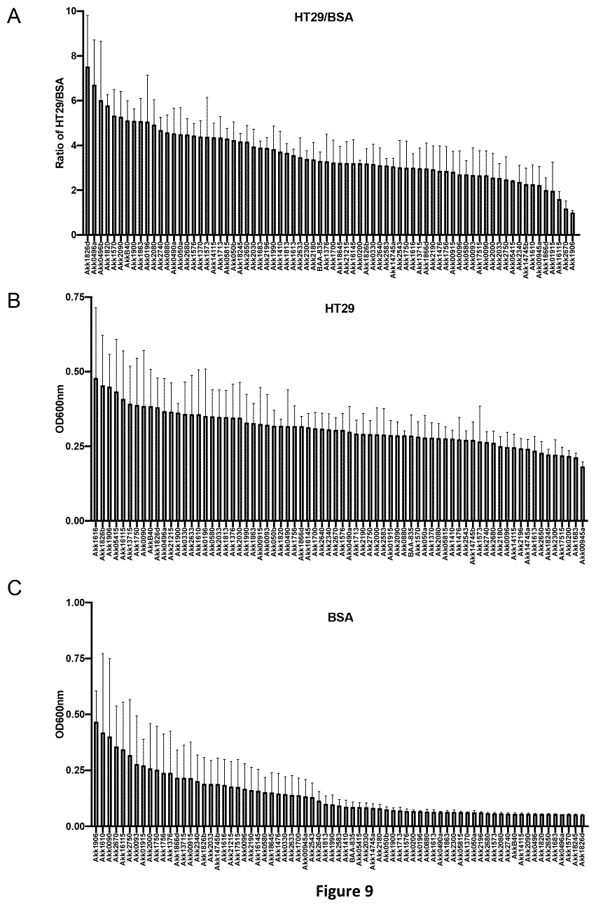

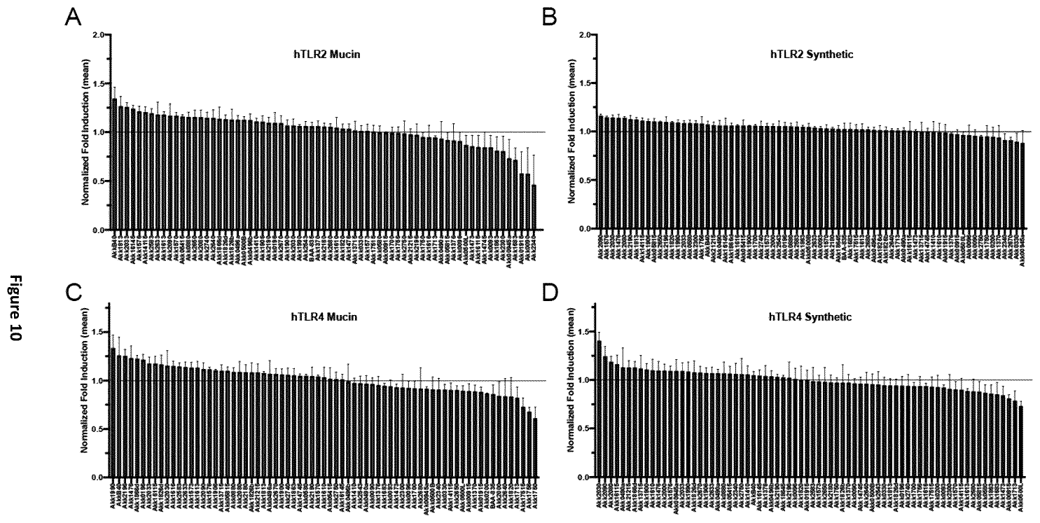

Adherence to epithelial cells: We determined if there are strain level differences in A. muciniphila isolates for their ability to attach to intestinal epithelial cells. Binding was assessed using HT29-MTX colonic epithelial cells grown on microtiter plates for 7 d past confluence, a stage at which they start secreting mucins. As a control for non-specific binding to biotic surfaces we used bovine serum albumin (BSA) coated plates. Adherence was monitored either microscopically or by following the outgrowth of A. muciniphila after addition of synthetic medium to wells. As previously reported, A. muciniphila strain Muc^{T} bound to human colonic cells (20), albeit at low levels. Our A. muciniphila isolates displayed a broad range of binding affinities to HT29 monolayers (FIG. 2C, FIG. 9AB and Table S3), as compared to BSA coated plates. Strains also differed in their binding to BSA-coated plates alone (FIG. 9C).

Mucin fermentation: The major end products of mucin metabolism by A. muciniphila are acetate and propionate, with minor amounts of succinate and 1,2-propanediol (16, 21). The ratio of acetate to propionate generated is influenced by how simple sugars are metabolized and the availability of vitamin B₁₂ to activate methylmalonyl-coA and generate propionate (16). We measured SCFAs produced by the various Akkermansia strains after growth in mucin by gas chromatography. All A. muciniphila isolates produced acetate/propionate at a ratio of 1.21-1.47 after reaching stationary phase of growth in 0.5% mucin medium, which is in the range of what has been reported for the A. muciniphila Muc^{T} strain (21).

Oxygen sensitivity: Although A. muciniphila is an anaerobe, its growth can be stimulated by low levels of oxygen (22). The inventors predicted that the relative tolerance of A. muciniphila to oxygen may impact their abundance near epithelial surfaces or their resistance to oxidative stress during GI inflammation. The inventors tested the sensitivity of A. muciniphila strains to oxygen by exposing agar plates to ambient air for 12, 18 and 24 h before returning the plates to anaerobic conditions (FIG. 2D). We determined that there is a range of responses, with some strains being fairly tolerant to prolonged exposure to ambient oxygen (˜60% survival at 24 h), while others were extremely sensitive (<0.01% survival at 12 h) (Table S3).

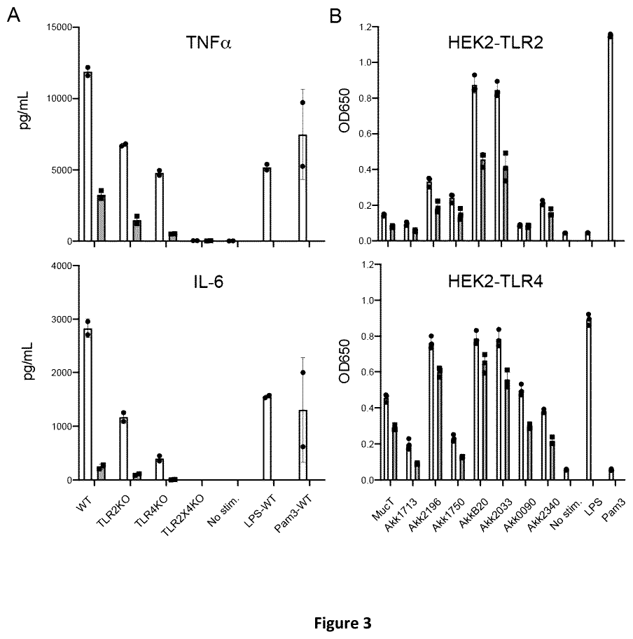

Activation of innate immune sensors: Human monocytes are activated by exposure to A. muciniphila Muc^{T} (23) and TLR2-dependent recognition is required for signal transduction events that strengthen barrier function in the GI tract (24). The inventors first tested what are the relevant TLRs involved in the recognition of A. muciniphila by stimulating bone marrow derived macrophages (BMDM) from wild type C57BL/6J mice and from tlr4, tlr2 tlr4, unc93b1, and tlr2 tlr4 unc93b1 knock out mice (25). Unc93b1^{-/-} mice are defective for the transport of TLRs that sense nucleic acids as well as the expression of TLR5, which recognizes flagellin, at the cell surface (26, 27). BMDM were incubated with A. muciniphila Muc^{T} at a ratio of 0.5 or 5 bacteria per cell for 6 h and the secretion of the cytokines TNF□ and IL6 was assessed (FIG. 3A). At the lowest dose of Akkermansia, the response of BMDM was exclusively dependent on TLR4. TLR2-dependent activation was only apparent when bacterial loads were increased by 10 fold. Consistent with previous findings (23), additional TLRs were not required for immune activation of mouse BMDMs.

The inventors further used HEK-TLR reporter cell lines to test the ability of A. muciniphila isolates to specifically activate TLR2 and TLR4. We used cell lines expressing TLR4 or TLR2 and its co-receptors TLR1 and TLR6, which respond to known TLR2 ligands (28). A. muciniphila strains were grown in mucin or synthetic medium, incubated with reporter cell lines expressing TLR2 or TLR4 for 16 h at a ratio of 1 or 5 bacteria per HEK-TLR cell, and levels of TLR-dependent secreted alkaline phosphatase (sAP) was measured 16 h post stimulation (FIG. 3B and Table S3). A subset of strains induced TLR2 or TLR4 activation consistently above or below the mean of all A. muciniphila isolates tested (Table S3 and FIG. 10). The induction of TLR2 and TLR4 reporters was higher for bacteria grown in synthetic medium as compared to mucin medium (FIG. 10).

### Comparative Genomics Suggest that Phylogroup-Specific Genetic Determinants regulate A. muciniphila Replication in Mucin and Aerotolerance

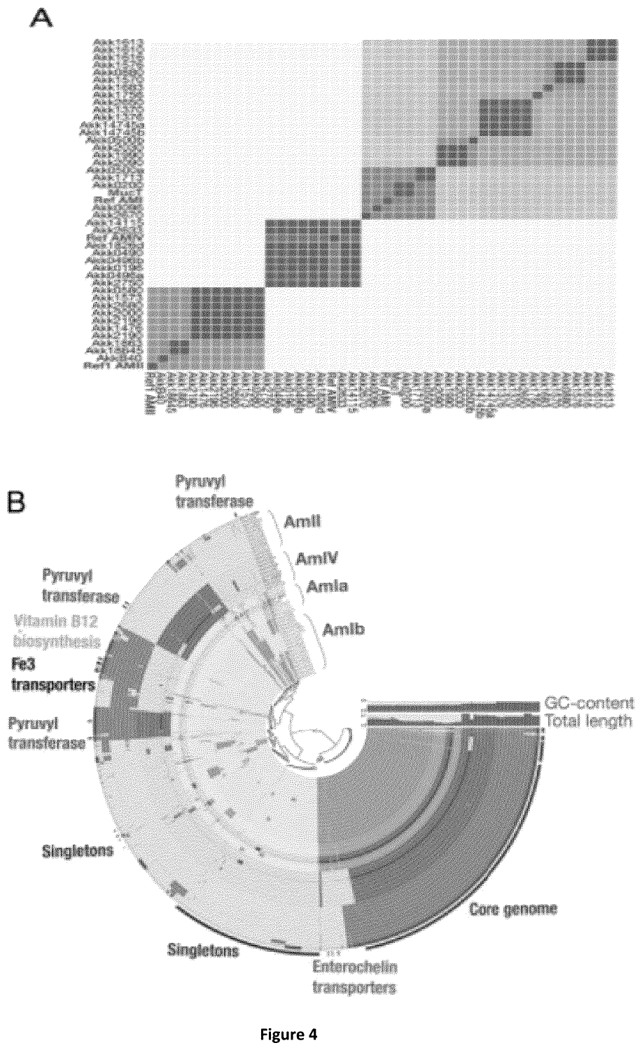

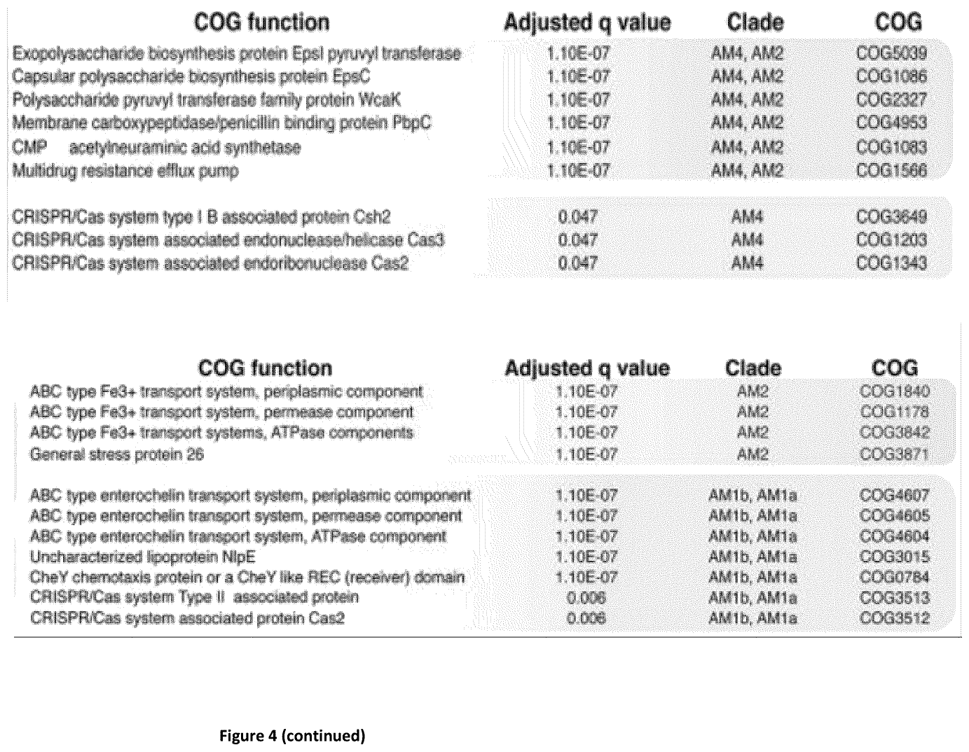

Of the four A. muciniphila phylogroups (clades AmI-IV) the most studied strain (Muc^{T}) is a representative of phylogroup AmI (16, 17). To assess the distribution of A. muciniphila phylogroups in the POMMS cohort, we sequenced the genomes of 43 isolates. A comparative analysis of these genomes indicated that we have representatives of phylogroups AmI, AmII and AmIV, but not AmIII (FIG. 4). We designed phylogroup-specific primers (Table S4) to genotype the isolates whose genomes had not been sequenced and determined that AmI (41/71) was almost twice as prevalent as AmII (22/71) and AmIV (8/71). Based on whole genome comparisons of AmI members, we propose that AmI can be further subdivided into two related subclades (Ia and Ib) at a threshold of 96% average nucleotide identity (ANI). Genomes ranged in size from 2.6-3.3 Mb, and phylogroups AmII and AmIV were consistently larger than the AmI genomes (FIG. 4AB and Table S3).

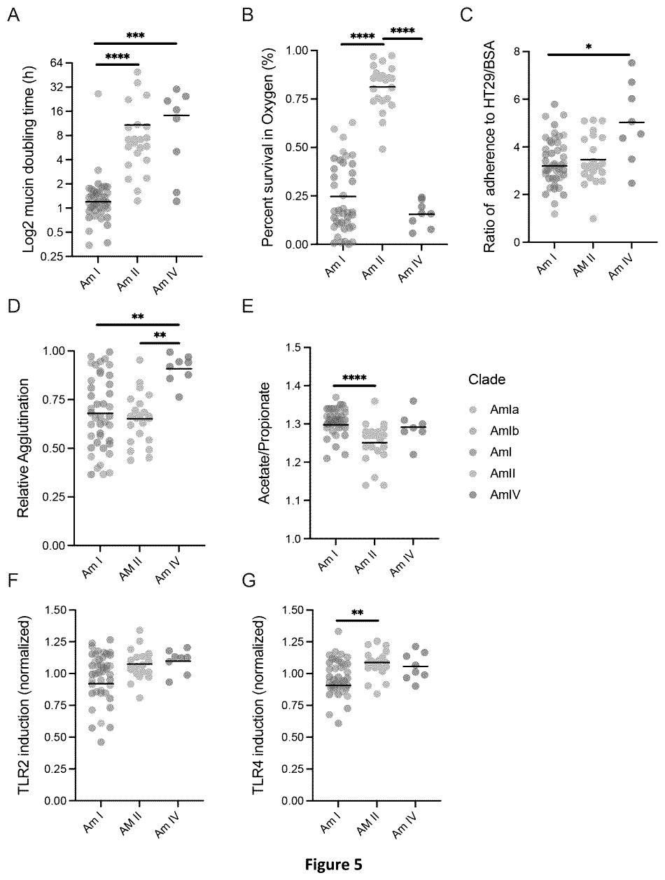

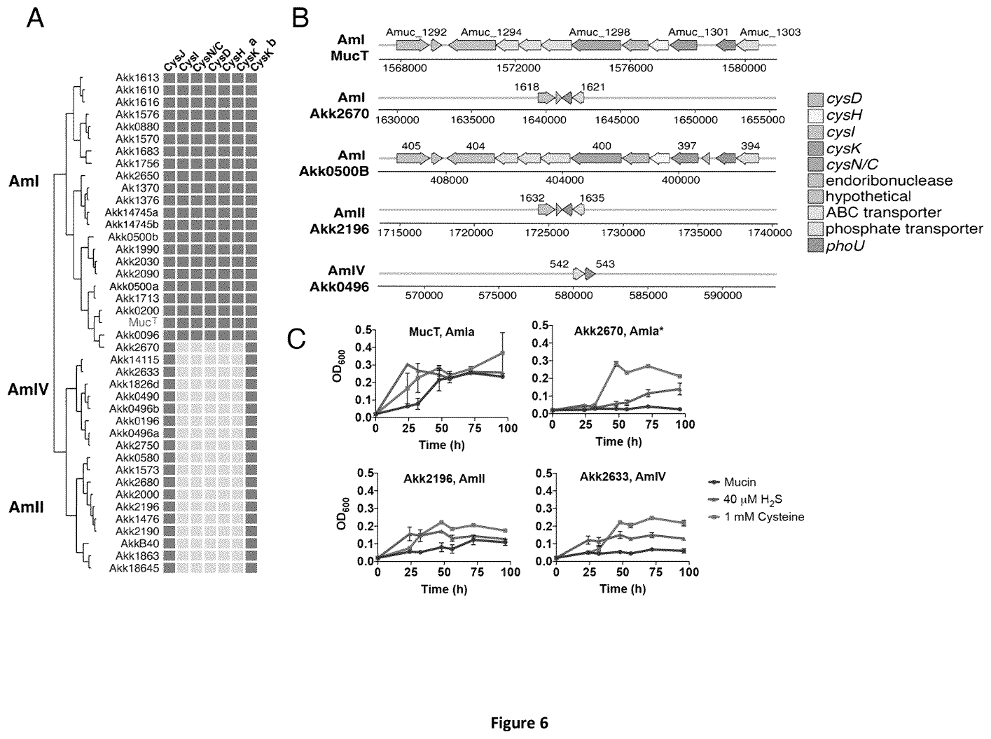

We determined that several phenotypes segregated by phylogroup (FIG. 5). AmI strains displayed rapid doubling times while most members of AmII and AmIV grew slowly (FIG. 5A). Strains also differed in their sensitivity to ambient oxygen, with strains AmII being resistant and AmIV very sensitive (FIG. 5B). Differential oxygen sensitivity was also observed within phylogroup AmI, with AmIb strains being highly sensitive to exposure to air while AmIa strains displayed intermediate resistance. AmIV strains had higher adherence to epithelial cells and displayed a greater propensity to aggregate when grown in mucin medium (FIG. 5C-D). We also saw a small drop in the ratio of acetate/propionate fermentation end products of mucin fermentation for AmII strains (FIG. 5E), which we postulate is because they synthesize vitamin B₁₂ (16) and hence generate more propionate as vitamin B₁₂ in the growth medium becomes limiting.

For the stimulation of HEK-TLR reporter cell lines, AmII and AmIV strains were more stimulatory for both TLR2 and TLR4 than AmI strains (FIG. 5F-G) but it is unclear if this is simply a reflection of their enhanced binding properties to cell surfaces. AmI strains displayed a broad range of activation of TLR reporter cell lines, and while there was a trend for AmIb strains to be more stimulatory, particularly for TLR4, the differences did not reach statistical significance given the relatively low number of AmIa isolates in our strain collection.

To identify genes that may contribute to these phenotypes, we analyzed the pangenome of our A. muciniphila strains and identified 4982 total gene clusters, with 1647 core gene clusters found in all genomes, and 506 gene clusters found only in single genomes (FIG. 4A). We found several phylogroup specific gene groups that may contribute to phenotypic variation (FIG. 4B-C and Table S3). For example, all members of phylogroups AmII and AmIV were predicted to encode distinct capsule and exopolysaccharide genes that were absent in AmI strains. These included putative capsular polysaccharide (CPS) biosynthesis proteins EpsC and EpsI, a CMP-N-acetylneuraminic acid synthetase, and a capsule modifying enzyme, polysaccharide pyruvyl transferase WcaK (29). The AmI isolates have a single Cps locus, while AmII and AmIV genomes contain two additional loci that are largely conserved among these phylogroups and map to similar regions in the chromosome. This suggests common capsule types are present in the phylogroup AmII and IV strains. Phylogroup AmIV also codes for DltB, an enzyme typically involved in modification of lipoteichoic acid in Gram-positive bacteria but that can also modify lipopolysaccharides in some Gram-negative bacteria (30). Conversely, members of phylogroup AmI had additional chemotaxis genes, cytochrome c biosynthetic genes, and code for a quality control sensor protein for outer membrane biogenesis, NlpE (31).

The phylogroups also displayed differences in iron acquisition systems. Anaerobic conditions favor reduced ferrous iron (Fe²⁺) and aerobic conditions the oxidized ferric iron (Fe³⁺) (32). Although all genomes had a ferrous iron transport system consisting of FeoAB genes (Amuc_1088, Amuc_1089, Amuc_1090 in Muc^{T}), phylogroups AmI and AmII encoded additional mechanisms to acquire ferric iron. Members of AmI had multiple enterochelin transporter gene groups, suggesting that they might use siderophores to scavenge ferric iron. Members of phylogroup AmII also have predicted ferric iron transporters, although these appear to be distinct from the gene groups in phylogroup AmI. In contrast, phylogroup AmIV lacks canonical mechanisms for ferric iron acquisition. Phylogroup AmIV is highly sensitive to ambient oxygen, and it is plausible that defects in iron acquisition or the absence of the oxidative stress protection associated with siderophores (33, 34) may contribute to this phenotype. Additional contributors to differences in oxygen sensitivity include a LexA repressor, indicative of an SOS system, that is present in all phylogroup AmI and AmII strains.

The A. muciniphila strains were predicted to encode approximately 27 glycoside hydrolase (GH) enzyme families, but they varied in the abundance of GH families among the phylogroups, particularly in AmIV genomes (FIG. 3B). GH97 enzymes, which comprise glycoamylases such as B. thetaiotaomicron SusB (35) were detected in all strains except for AmIV. Similarly, AmIV genomes had fewer GH110 enzymes, a group of galactosidases capable of cleaving blood group B antigens (36). Conversely, AmIV strains were enriched for GH29 and GH95 L-fucosidases, which could potentially cleave the terminal fucose residues that decorate mucin and human milk oligosaccharides (37). In humans, both ABH antigens and fucose modifications are more prevalent in ileal mucins, with potential implications for the relative localization of Akkermansia strains in the GI (38).

Another function with phylogroup-specific differences are the CRISPR/cas systems. While only a few of the phylogroup AmII genomes had CRISPR gene clusters, putative CRISPR/cas genes were detected in some AmI and in all AmIV genomes. The class of CRISPR system may be phylogroup-specific since AmIV strains were predicted to encode genes found in type I-B CRISPR/cas systems, while AmI strains were predicted to have genes associated with type II systems (Amuc_2008, Amuc_2009, Amuc_2010 in Muc^{T}) (39, 40), although it is not clear if these represent complete, functional systems. In some instances, the CRISPR genes are located close to predicted phage genes, possibly indicative of horizontal genes transfer.

Phylogroups AmII and AmIV are Deficient for Reductive Sulfur Assimilation

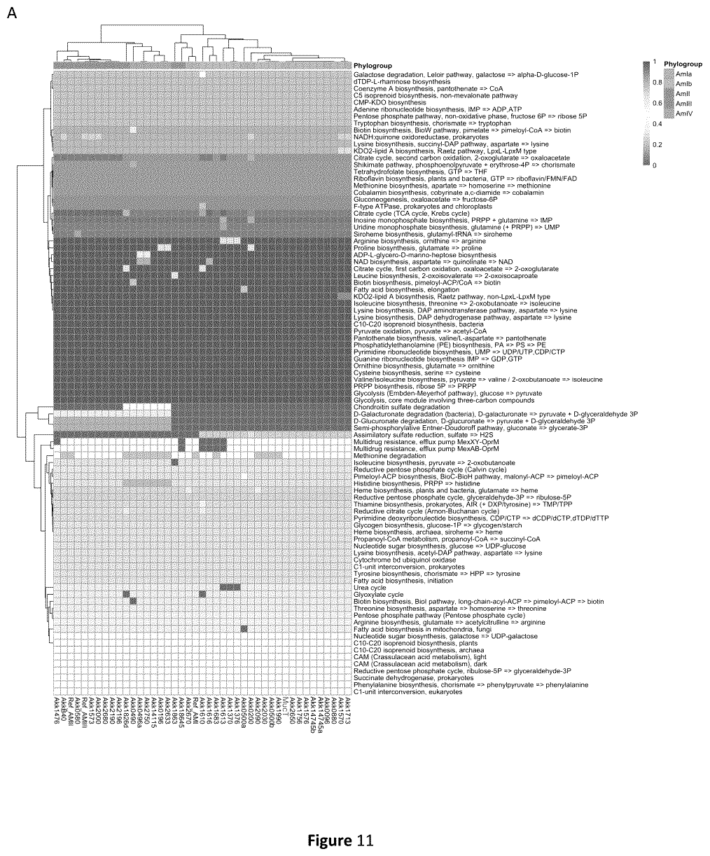

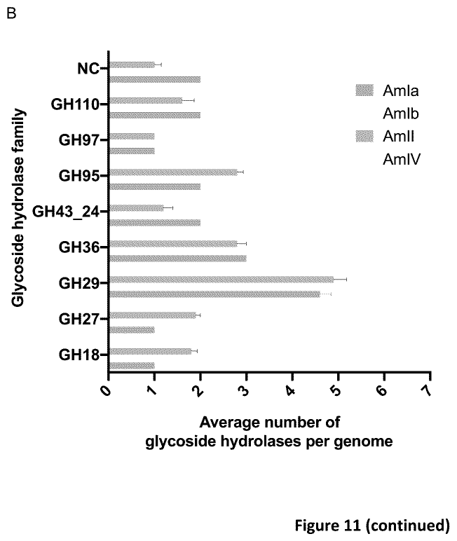

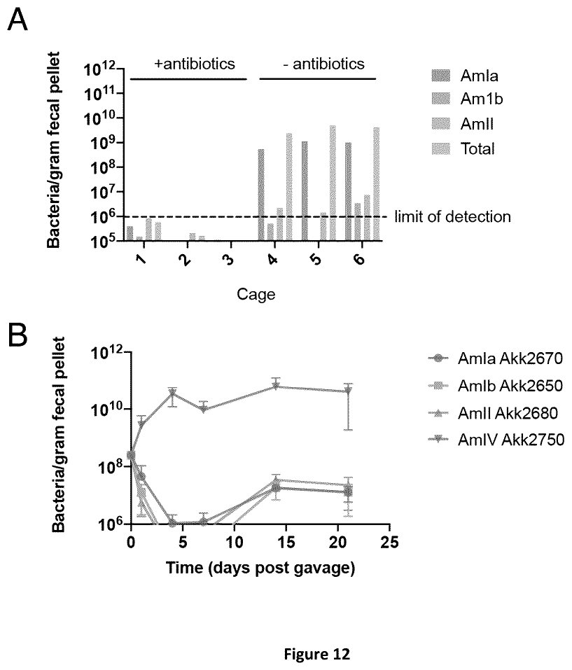

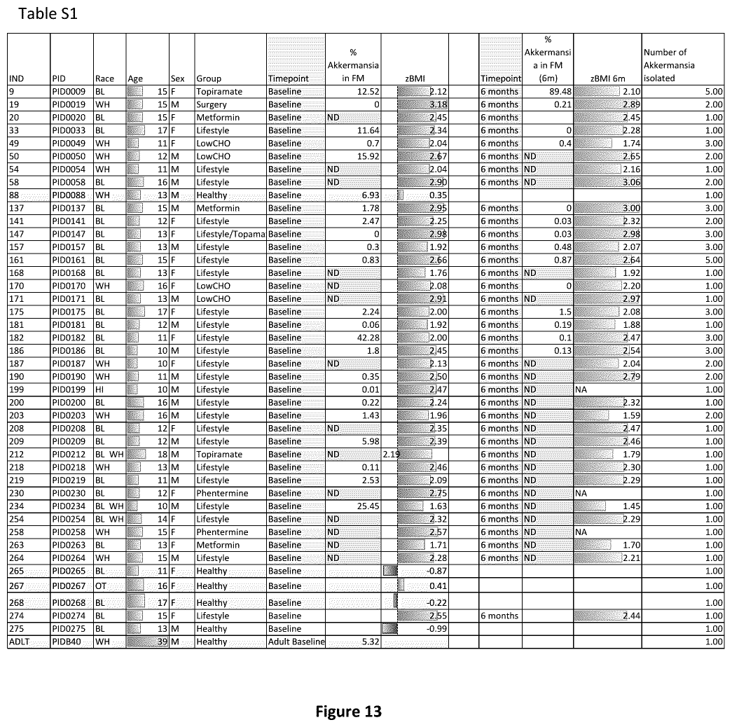

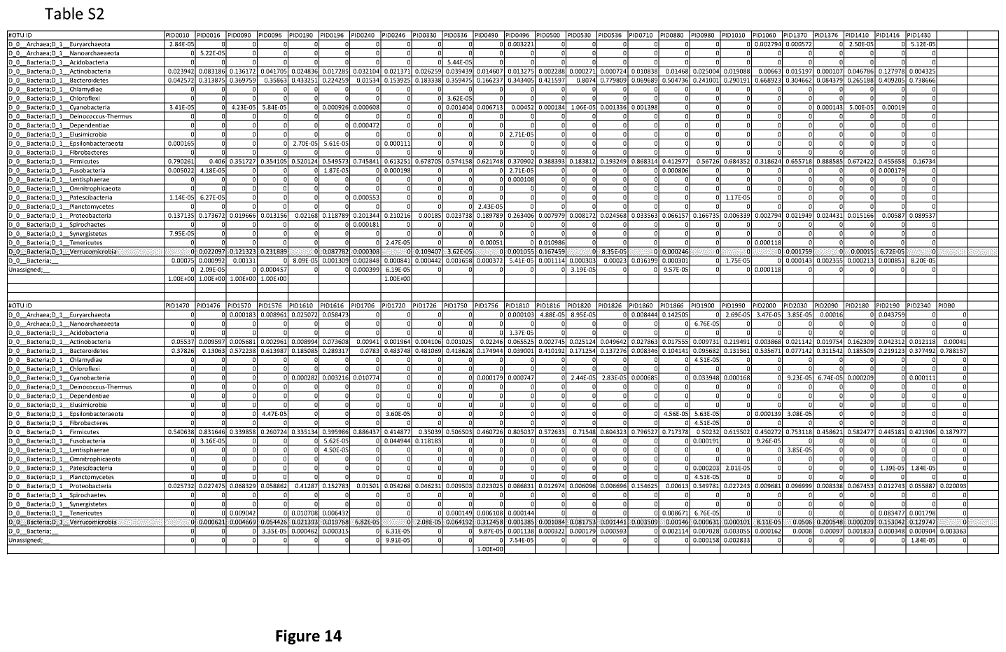

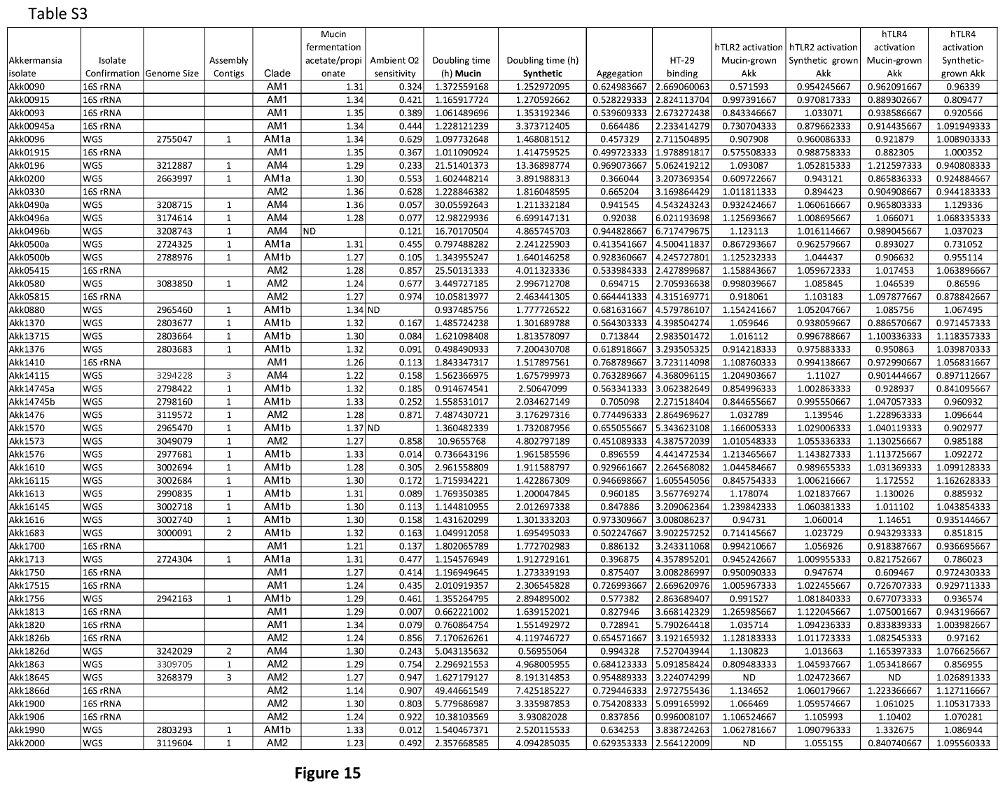

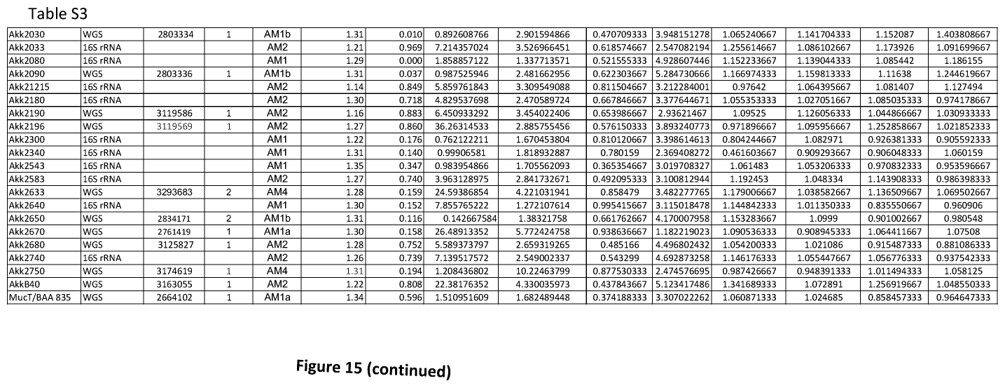

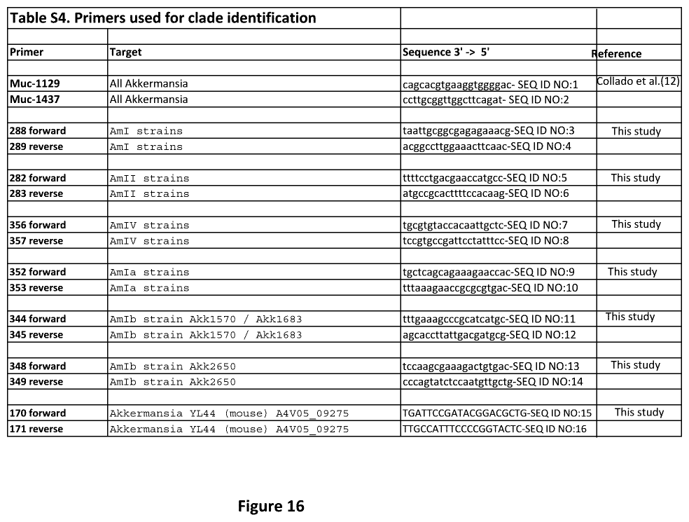

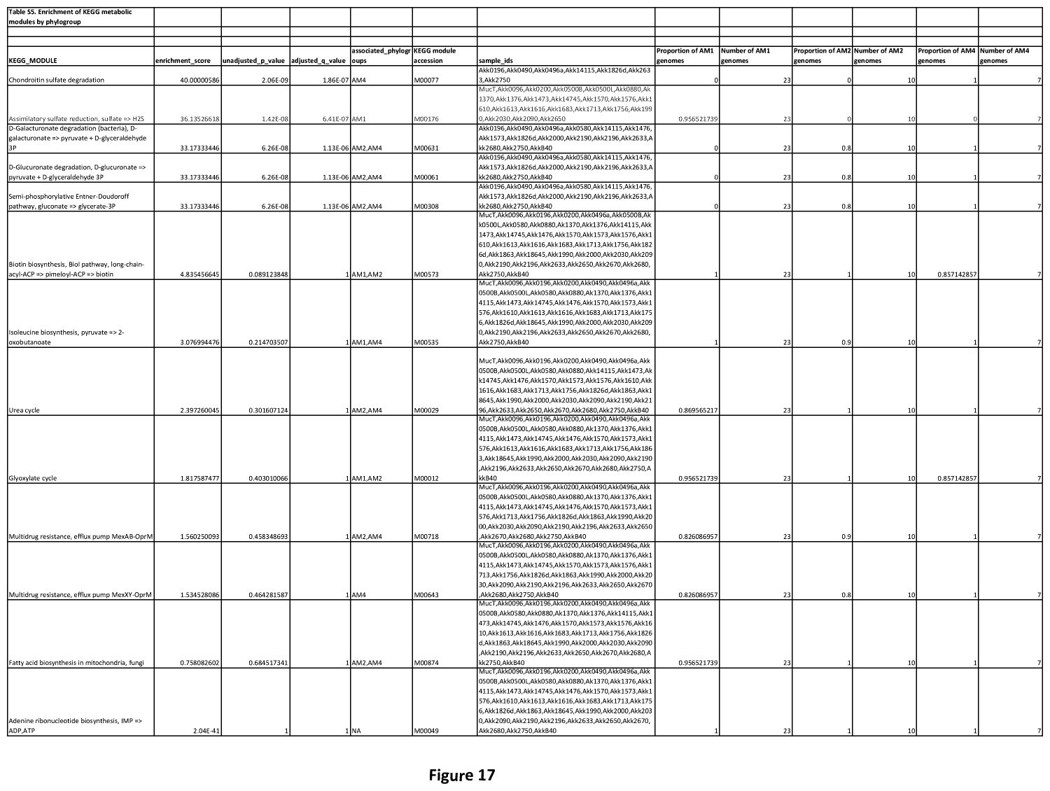

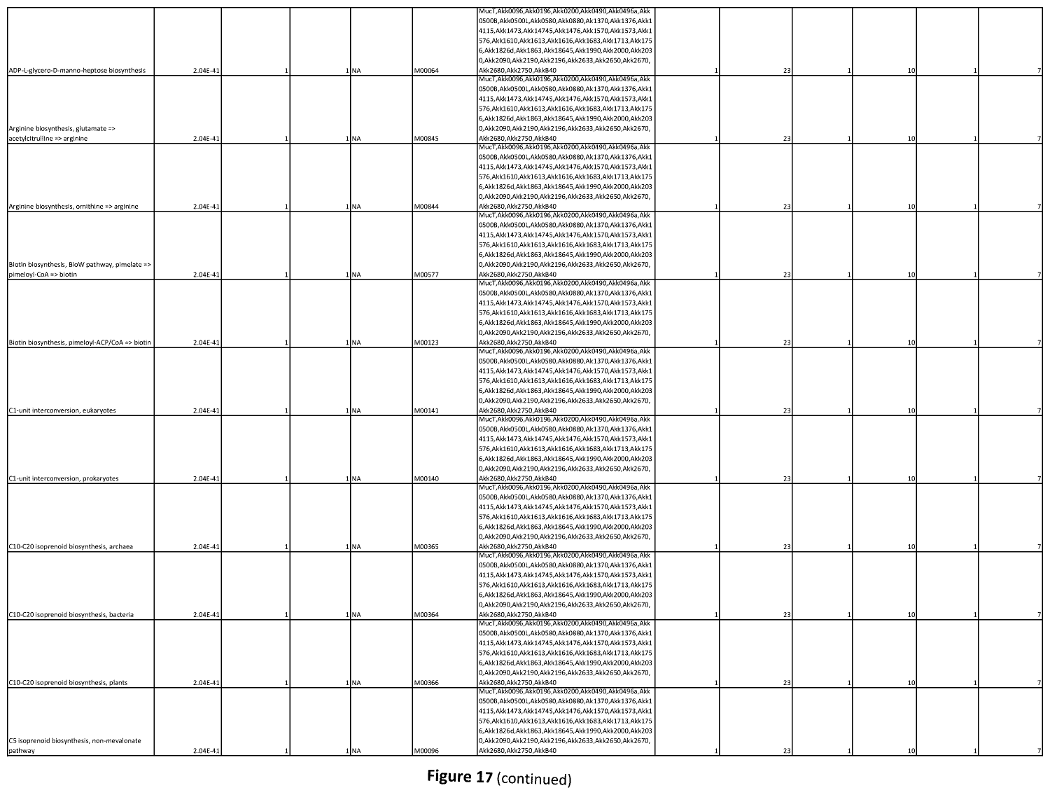

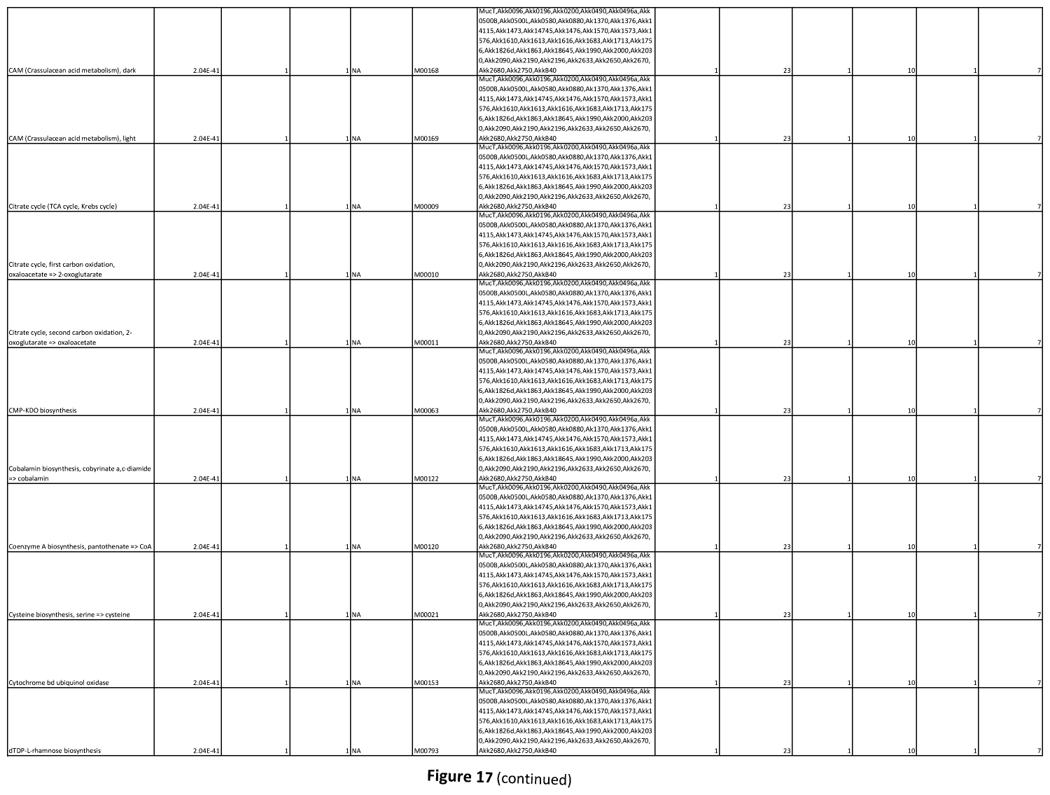

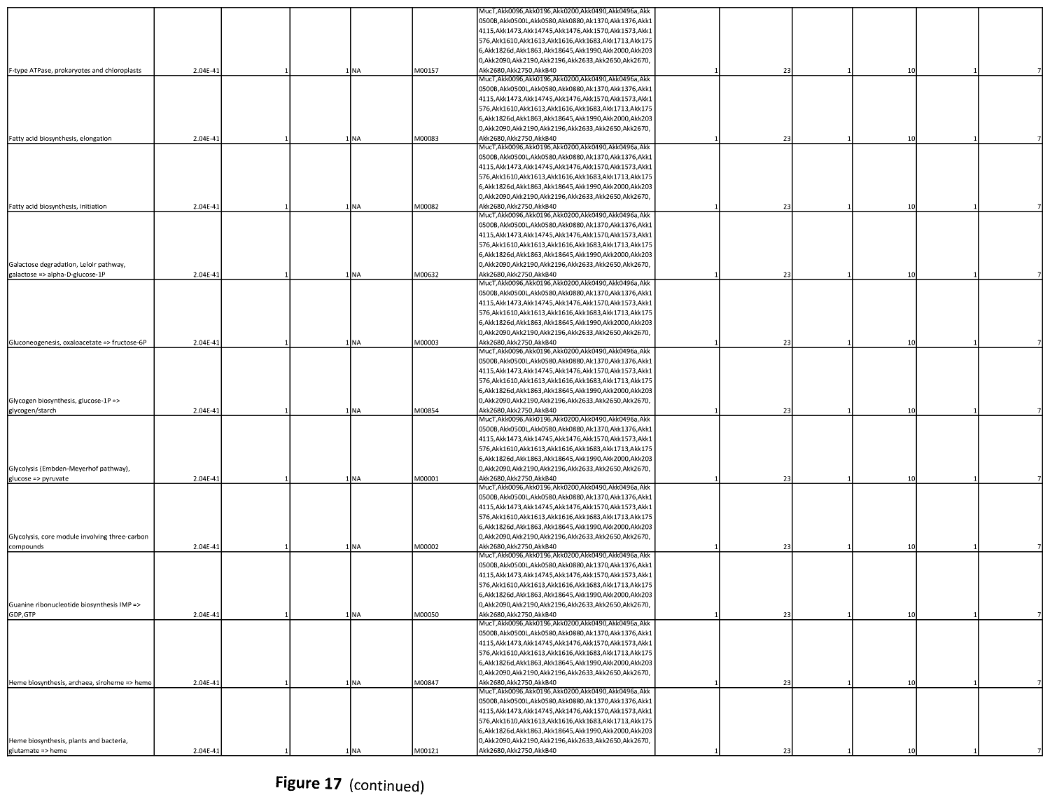

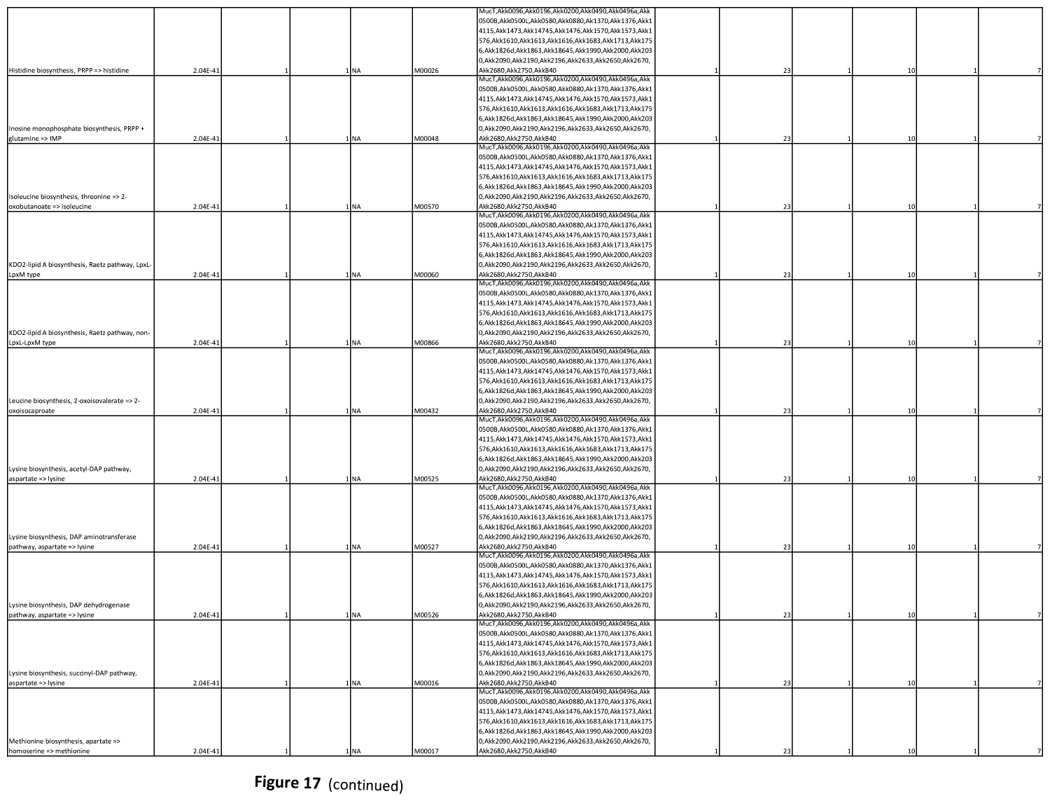

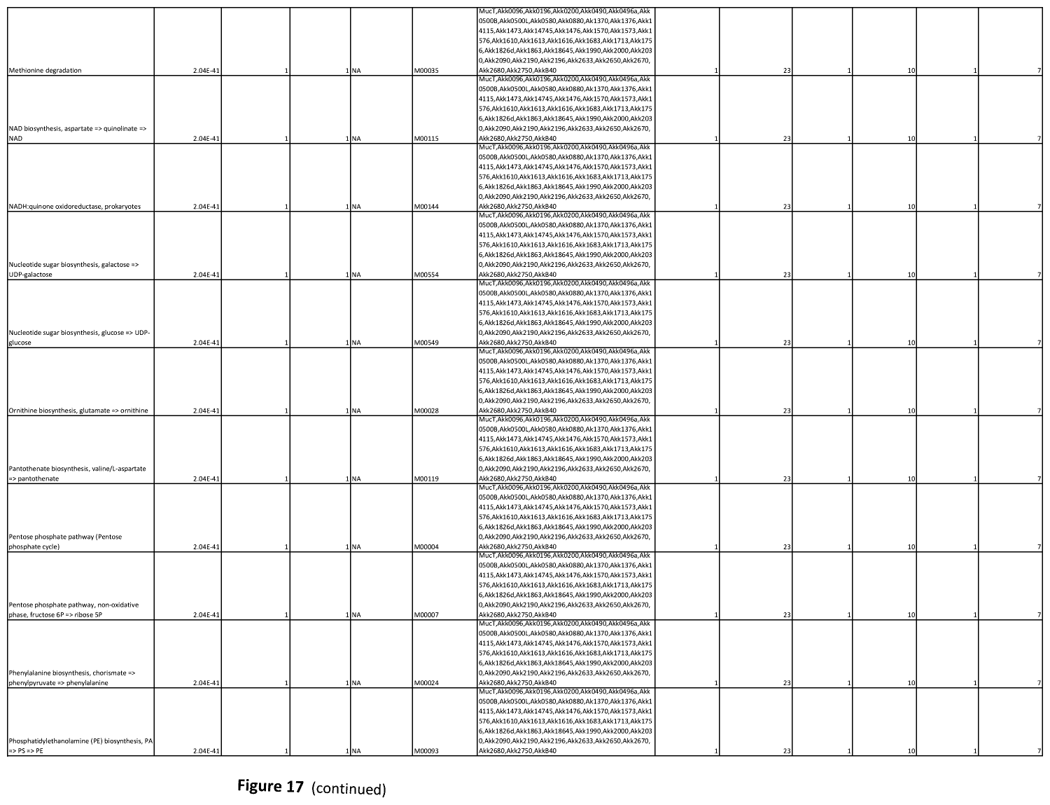

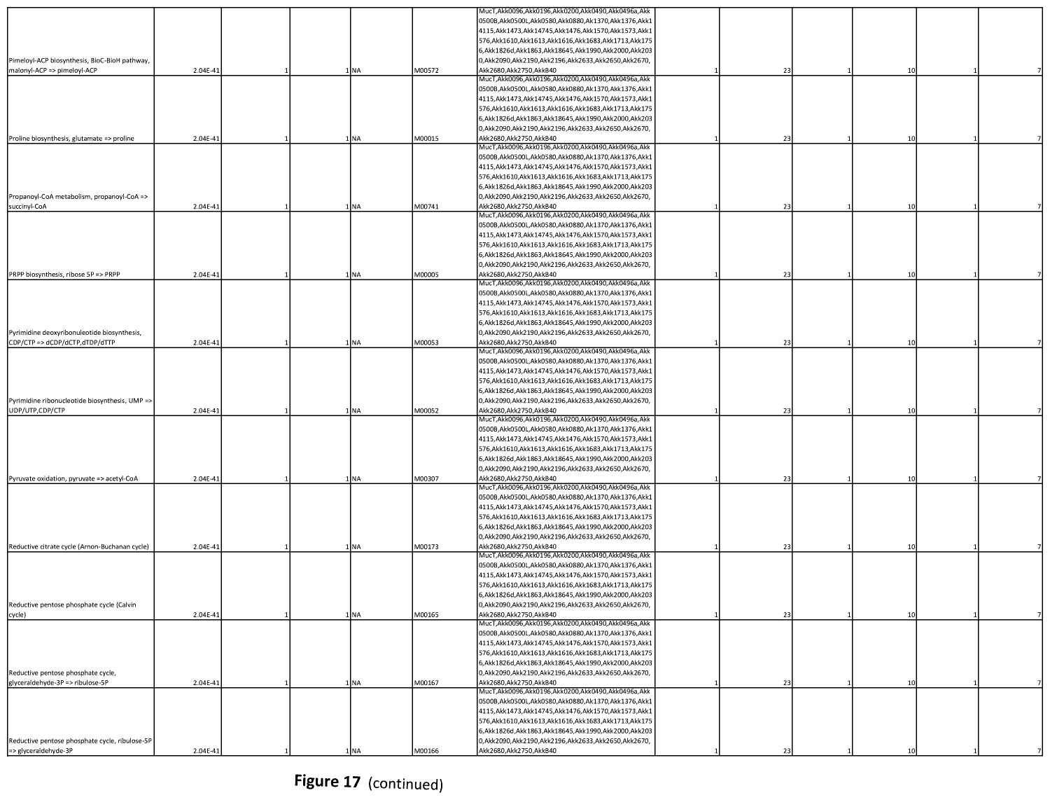

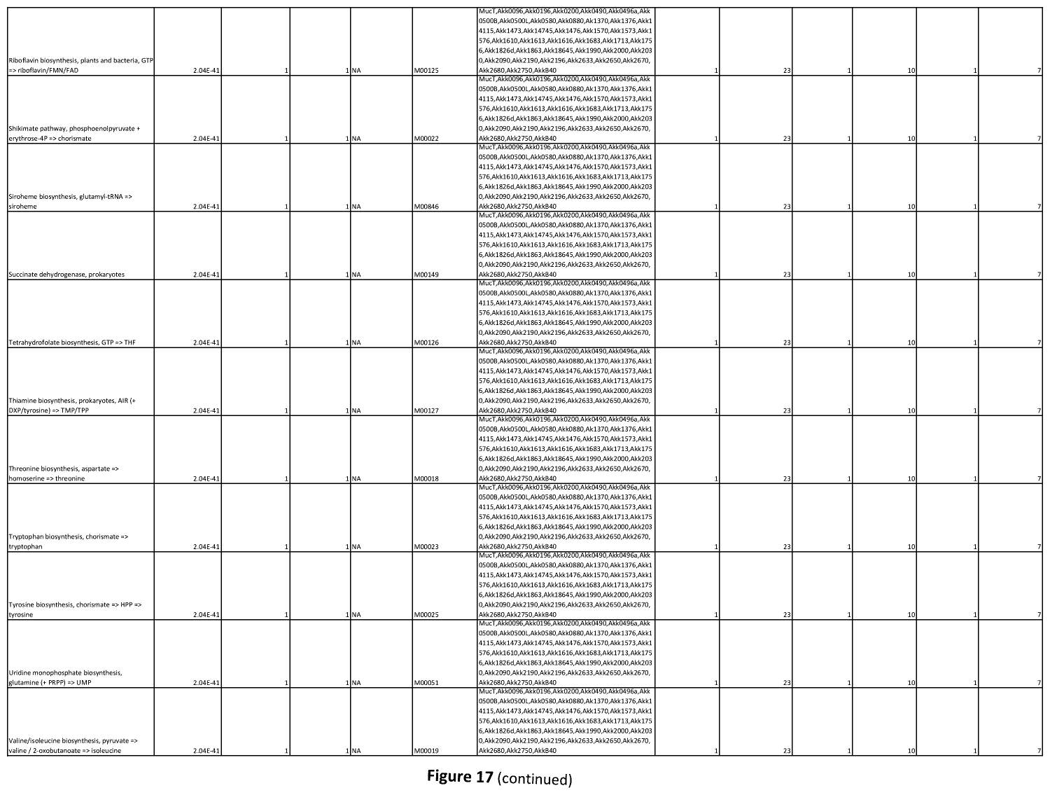

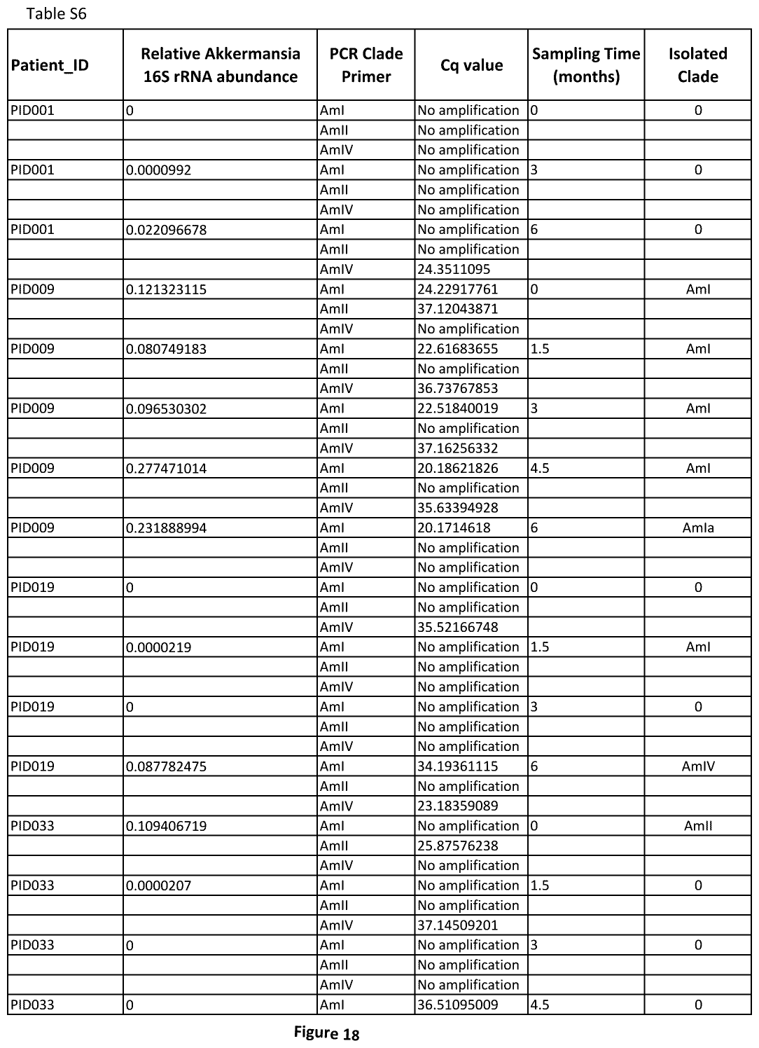

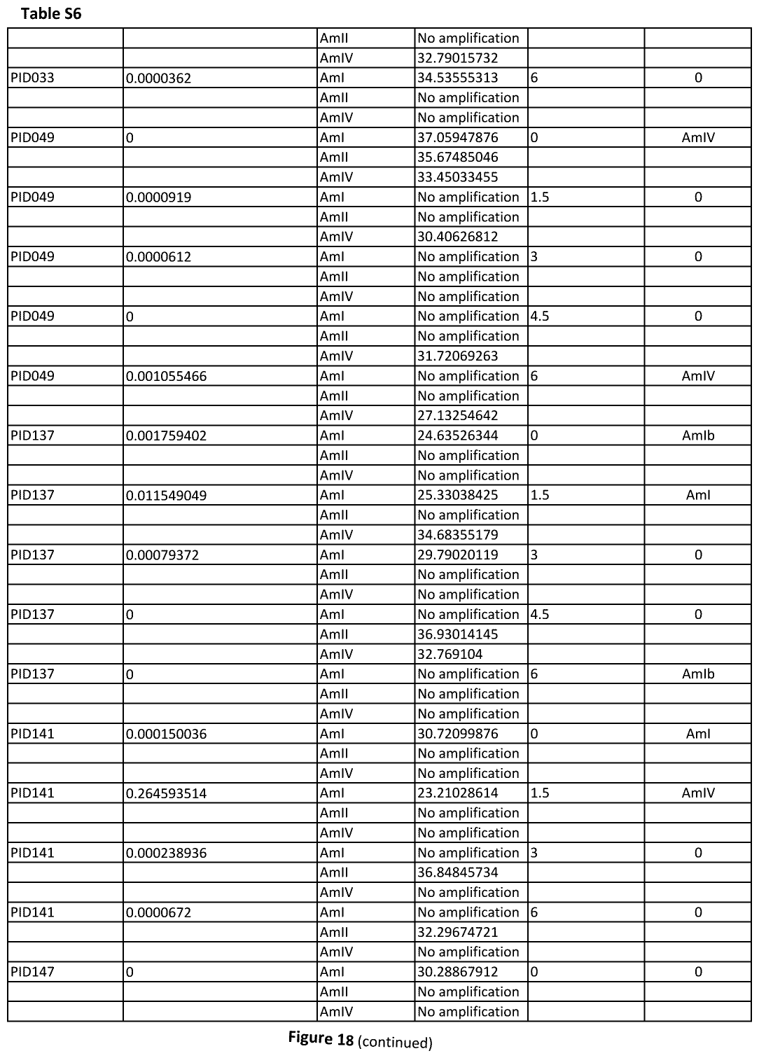

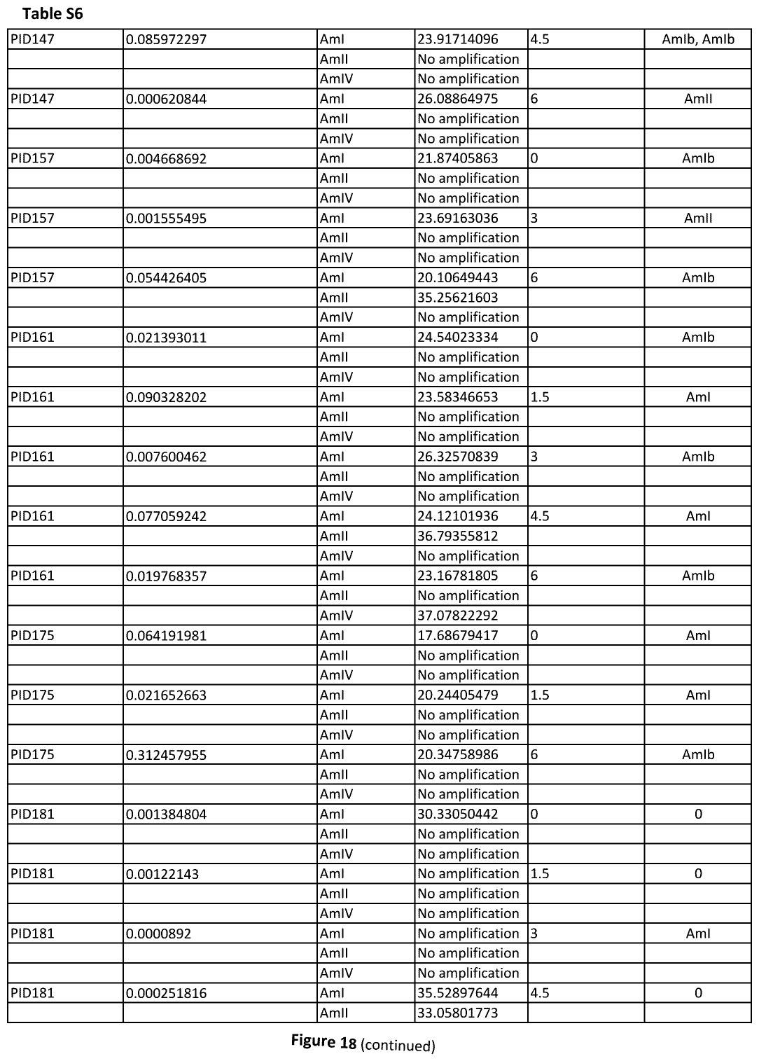

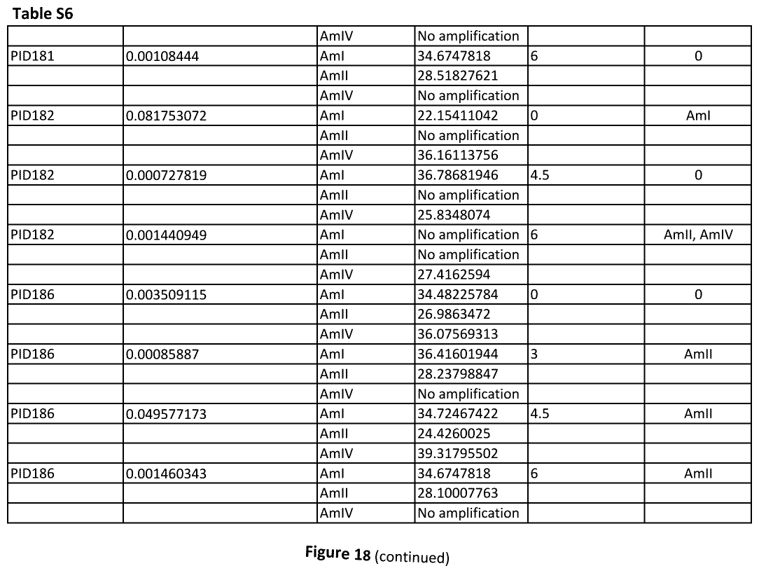

Analysis of the metabolic capabilities of the strains based on genomic sequences revealed additional predicted phylogroup-specific features (FIG. 6 and FIG. 11A). For instance, metabolic enrichment analysis showed that genes required for assimilatory sulfate reduction (ASR) are significantly enriched in phylogroup AmI (adjusted q-value 6.14E-7) (Table S5), but absent in AmII and AmIV isolates (FIG. 11A). Enzymes in the ASR pathway reduce sulphate to hydrogen sulfide for the synthesis of sulfur containing molecules such as cysteine and methionine. In the canonical ASR pathway, sulfate is first reduced to adenosine phosphosulfate (APS) by ATP sulfurylase (CysN), followed by the formation of phosphoadenosine phosphosulfate (PAPS) by APS kinase (CysD), which is further reduced to sulfite by PAPS reductase (CysH), and finally to H₂S by sulfite reductase (CysI/J) (41). H₂S is a substrate for cysteine synthase (CysK) to generate cysteine. In the Muc^{T} strain, ASR genes are clustered in a single locus (Amuc_1294 to Amuc_1301), with the exception of a CysJ homolog (Amuc_0631) and a second cysteine synthase (Amuc_2014). The locus also included a potential inner membrane sulfide permease (Amuc_1295), an ABC transporter related ATP-binding protein (Amuc_1296), and a substrate binding protein (Amuc_1297) (FIG. 6A and 6B).

AmII and AmIV strains lacked the ASR gene cluster found in Muc^{T} but retained distally encoded homologs for CysJ and the Amuc_2014 cysteine synthase (CysK-b) (FIG. 6A). All AmI strains were predicted to perform ASR except for the strain Akk2760 (FIG. 6A and FIG. 11A), which lacked the entire ASR locus (FIG. 6B). Since Akk2670 grew very poorly in mucin medium, with a growth rate comparable to that of AmII and AmIV isolates (FIG. 5A and Table S3), the inventors hypothesized that the inability to generate reduced sulfur may be a limiting factor for their growth on mucin in vitro. To test this, representative strains were grown in mucin medium with or without the addition of cysteine or sodium hydrosulfide as a source of H₂S. While cysteine and H₂S shortened the lag time for the growth of the Muc^{T} strain it did not affect the maximal biomass achieved (FIG. 6C). In contrast, cysteine significantly enhanced the maximal growth of the predicted ASR-deficient AmIa strain Akk2670, the AmII strain Akk2196 and the AmIV strain Akk0496 (FIG. 6C). These findings suggests that A. muciniphila strains benefit from the addition of reduced sulfur when grown in mucin and that the growth of ASR-deficient phylogroups is significantly enhanced by the addition of cysteine or H₂S.

AmIV Strains Outcompete other Phylogroups in a Murine Colonization Model

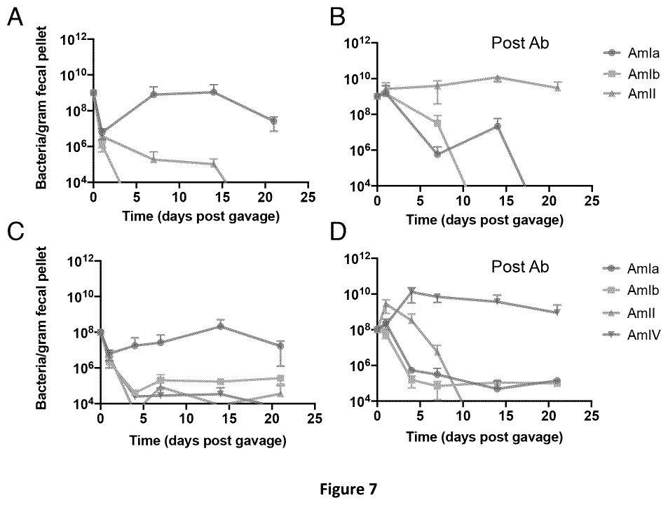

To determine if A. muciniphila phylogroups varied in how they colonize animals we assessed the ability of representative isolates of each phylogroup to compete in the mouse GI tract. Mice housed in our vivarium are naturally colonized by a mouse Akkermansia strain, which belongs to the AmIa phylogroup (not shown). We first challenged these mice with human strains representing phylogroups AmIa, AmIb, and AmII (FIG. 7A) and their abundance in fecal pellets was monitored over time by qPCR. We found that the human AmIa, AmIb, and AmII strains failed to engraft with the endogenous mouse A. muciniphila and associated microbiota which provided colonization resistance against introduction of additional Akkermansia strains. We cleared mice of its Akkermansia and other microbes with a 14 day regimen of tetracycline and repeated the competition experiments with the same cocktail of human A. muciniphila phylogroup representatives. Under these conditions, the AmII strain (Akk0580) became the dominant phylogroup by 20 days post inoculation (FIG. 7B).

Next, the inventors competed strains representing all four phylogroups: AmIa, AmIb, AmII, and AmIV. As with the previous experiment, the human A. muciniphila isolates failed to colonize mice with an intact microbiota (FIG. 7C). However, pretreatment with antibiotics enabled engraftment of the newly introduced strains, but this time the AmIV strain (Akk0490) became the predominant phylogroup (FIG. 7D). These findings were recapitulated with a second set of representative strains, with the AmIV isolate (Akk2750) rapidly becoming the dominant phylogroup (Fig S4). These findings suggest that AmIV strains, despite their slow growth in mucin medium and high sensitivity to oxygen, overtake other phylogroups in the GI tract when placed in direct competition in mice whose microflora had been depleted.

### Evidence for Phylogroup Exclusion and Switching in Patients Colonized with A. muciniphila

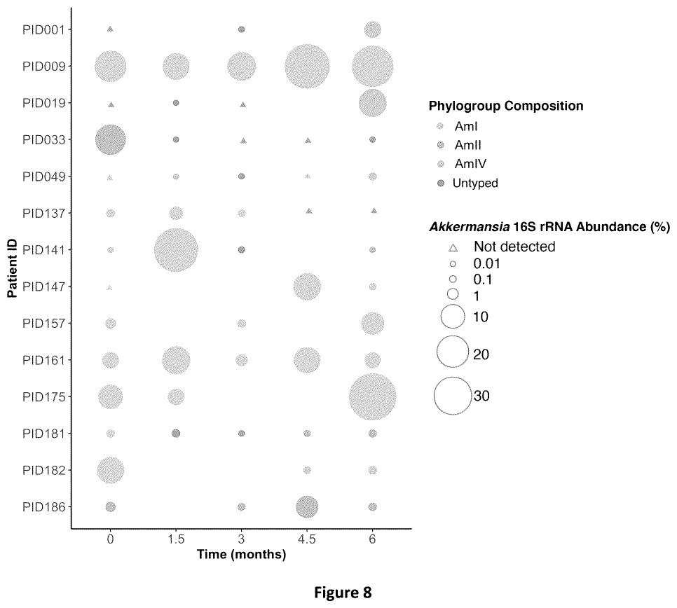

Because AmII and AmIV strains prevented AmI strains from establishing themselves when placed in direct competition in mice, the inventors hypothesized that A. muciniphila phylogroups occupy the same ecological niche and that AmII and AmIV strains have a selective advantage in the GI tract. To assess the natural distribution of major phylogroups in humans, we used phylogroup-specific primers (Table S4) to quantify AmI, II, and IV strains in fecal samples collected from 14 patients at baseline and at 1.5 month intervals after enrolling in POIVINIS (FIG. 8). In stool samples where a Glade-specific signal could be detected above background, we found dominance of a single major phylogroup regardless of the total relative abundance of Akkermansia in that sample. Surprisingly, in three instances the phylogroup of the A. muciniphila strains cultured (Akk1476, Akk1573, and Akk14115) did not match the dominant phylogroup identified by qPCR in the stool sample, and in one patient we isolated both AmII and AmIV strains (Akk1826b and Akk1826d) from the same fecal material, even though AmIV was the only strain identified by qPCR. This discrepancy between culture-based isolation and molecular quantification may reflect biases in culturing efficiency based on differences in oxygen tolerance that could impact the viability of Akkermansia during the collection, transport and handling of stool samples or differences in doubling times in porcine mucin medium during the serial enrichment process. Nonetheless, these findings suggest that despite the predominance of any one Akkermansia phylogroup in the GI tract, that additional strains can be present and viable despite being below the levels of detection by molecular methods.

In addition to evidence for co-existing of minor clades, we observed major changes in the overall abundance of Akkermansia phylogroups within the same patient. In 11 of 14 of patients with multiple sampling over a 6 month period, either the relative amount of Akkermansia fluctuated significantly (>100 fold) among samples or the identity of the phylogroups switched (FIG. 8 and Table S6). For instance, three patients (PID141, PID181 and PID182) switched their predominant phylogroup from an AmI strain to either AmII or AmIV strains by 6 months. In two patients that underwent bariatric surgery (PID001, PID019) both converted from almost undetectable levels of Akkermansia at baseline to a phylogroup AmIV dominant microbiota by the 6 month timepoint. It is unclear if the emergence of new dominant phylogroups represents blooms of preexisting low abundance strains or new colonization events.

### DISCUSSION

Obesity is a multifactorial disease influenced by host genetics, diet, behavior, and the microbial ecosystems that populate the human GI tract. Diet remains a key driver of microbial composition and the phenotypes associated with these resident microbial communities strongly influence their impact on the immunological and metabolic health of their host (42). However, some bacterial species seem to play an oversized role on the health of their hosts. For instance, A. muciniphila has emerged as a potential probiotic since its abundance in the GI tract positively correlates with decreased incidence of metabolic disease, obesity and diabetes (9, 10).

There is a growing recognition that there's a great diversity of Akkermansia strains and species. A pangenomic analysis of Akkermansia genomes revealed four A. muciniphila phylogroups (16) and an analysis of >1000 Akkermansia genomes reconstructed from metagenomic sequences from human samples across the world suggested the existence of up to four new species (43, 44). While metabolic capacities of different Akkermansia strains have been inferred based on genome annotations, experimental validation is largely lacking because isolates of these new phylogroups have either not been collected or characterized. A notable exception is a recent analysis of the vitamin B₁₂ biosynthetic properties of a member of the AmII phylogroup (16).

In this work, the inventors leveraged fecal samples collected as part of the POM1VIS interventional study for childhood obesity (18) to isolate Akkermansia strains that reflect the diversity of phylogroups present in both healthy and diseased states in children. Importantly, the availability of cultured strains enabled the phenotyping of each isolate to identify variances in traits that may impact the health of their hosts. The inventors cultured mucophilic bacteria from 123 fecal samples derived from 49 donors and isolated 71 new strains of A. muciniphila (Table S1 and S3). The relative abundance of Verrucomicrobia amplified sequence variants (ASVs) ranged from non-detectable to >30% of total sequences in children with obesity (Table S2).

Based on the genomes of 43 of new Akkermansia strains we determined that these isolates belonged to A. muciniphila phylogroups AmI, II and IV (FIG. 4). AmI strains constituted over half of isolates, which we propose should be subdivided into two subgroups (AmIa and Ib) based on an ANI cutoff of 96% among complete genome sequences and phylogroup-specific phenotypes, such as increased sensitivity to ambient O₂ for AmIb strains (FIG. 5A). In contrast, AmII strains were more resistant to ambient O₂ than all the other phylogroups. AmII strains were also more immunostimulatory for TLR2 and TLR4, although this was most apparent when bacteria were grown in mucin, as opposed to synthetic medium (FIG. 5F-G and FIG. 10). AmIV strains also displayed enhanced activation of TLR4 and TLR2 reporter cell lines which may reflect their increased binding to epithelial cells (FIG. 5C-D). Using BMDM we confirmed that TLR2 and TLR4 are the relevant pattern recognition receptors for the detection of A. muciniphila as previously reported (23), with TLR2 activation requiring a higher threshold for activation than TLR4 (FIG. 4A). TLR2-mediated enhancement of tight junctions in intestinal epithelia has been proposed as a mechanism to explain Akkermansia-dependent enhancement of gut barrier function (23). TLR2 forms heterodimers with TLR-1 or TLR6, with TLR2/1 being preferentially activated by triacylated lipopeptides from Gram negative bacteria and TLR2/6 by diacylated lipopeptides most commonly expressed on Gram positive bacteria (45, 46). Amuc_1100, a highly expressed pilus-like protein conserved among all phylogroups, has been proposed to be a major A. muciniphila TLR2 agonist (14, 23). We suspect additional TLR2 agonists exist given that A. muciniphila can stimulate HEK2923 cells expressing either TLR2/1 or TLR2/6 alone.

Although A. muciniphila is classified as a strict anaerobe, the growth of the reference Muc^{T} (AmIa) isolate is enhanced by nanomolar concentrations of O₂ (22). The ability to use oxygen as a terminal electron acceptor may provide A. muciniphila an advantage over other colonic bacteria when in proximity to epithelial surfaces. The closely related AmIb strains, however, are very sensitive to ambient oxygen even though the genomes are highly similar and also encode for the cytochrome bd complex, which is required to use O₂ as a terminal electron acceptor (22). The mechanism underlying this differential sensitivity to oxygen is not apparent from comparative genomics. In contrast, AmII strains' high resistance to oxygen may be linked to ferrous and ferric iron transport as genes encoding Fe³⁺ ABC transporters have expanded in AmII strains and iron transporter genes are highly expressed in A. muciniphila Muc^{T} when switched to aerated growth conditions (22). AmIV strains also display high sensitivity to oxygen, which may also be associated with decreased iron acquisition as this phylogroup lacks components of enterochelin transport system present in AmI strains.

Given the slow doubling times for both AmII and AmIV strains in vitro, we did not expect these strains to overtake AmI strains in antibiotic-treated mice. One possible explanation is the overrepresentation of capsular and exopolysaccharide gene clusters in AmII and AmIV phylogroups may provide protection from IgA and host-derived antimicrobial peptides. Unexpectedly, AmIV strains outcompeted AmII strains in mice, even though AmIV was expected to be more sensitive to oxygen present near colonic epithelium. Given these findings, it is clear that the complexity of the GI ecosystem makes it difficult to predict which in vitro phenotypes are most relevant for in vivo colonization of the GI tract.

A comparison of the predicted metabolic pathways in A. muciniphila strains indicated a clear absence of components of the assimilatory sulfate reduction (ASR) pathway in AmII and AmIV strains, and in one AmIa isolate (Akk2670) (FIG. 6). ASR is required to harvest sulfur from imported sulfate for the biosynthesis of amino acids. Akk2670 was unique among AmI strains in that it displayed a very slow growth rates in gastric mucin. This led us to postulate that the long doubling times for Akk2670, and AmII and AmIV isolates, reflect a reduced capacity to synthesize sulfur-containing amino acids. Although mucin contains sulfur in the form of cysteine and methionine in the protein backbone and terminally sulfated glycans (47), the cysteine and methionine content may not be sufficient to support rapid growth and sulfate cannot be used in the absence of a functional ASR pathway. The ASR pathway generates H₂S which is used to synthesize cysteine through condensation with O-acetylserine by cysteine synthase (48). Most AmI strains have two putative cysteine synthases (Amuc_1301 and Amuc_2014 in MucT), with Amuc_2014 being conserved in all Akkermansia (FIG. 6B). Cysteine also plays an essential role as a sulfur source for the biosynthesis of essential cofactors, vitamins, and anti-oxidants (48). Consistent with this prediction, the growth of Akk2670, AmII and AmIV strains is significantly enhanced by addition of exogenous cysteine or H₂S to mucin medium (FIG. 6C).

It is clear that the loss of ASR is not essential for GI colonization by A. muciniphila and may even enhance competitiveness given that sulfate transport and reduction is an energy intensive process (49, 50), particularly if other sources of reduced sulfur are available from the host or the microbiota. Potential sources of reduced sulfur in the GI tract include taurine, low levels of cysteine, and H₂S (51). For some members of the Bifidobacterium genus, cysteine auxotrophies are prominent, and at least in the case of B. bifidum cysteine auxotrophy cannot be rescued by supplementation of glutathione or taurine (52). Finally, several bacterial pathogens are cysteine auxotrophs, and even among species with complete ASR systems clinical isolates have been observed to spontaneously become cysteine auxotrophs (48). Thus there may be selective pressure for the loss of ASR genes in the presence of alternative reduced sulfur sources.

The loss of ASR in some A. muciniphila strains suggest that AmII and IV strain could be net consumers of any microbiota-derived H₂S especially under conditions where AmII and AmIV strains constitute a significant proportion of the entire microbiota (FIG. 8). If so, their localized detoxification of H₂S may contribute to some of the protection that has been ascribed to Akkermansia in the context of IBD and Crohn's disease (53-55). On the other hand, H₂S derived from the breakdown of cysteine by intestinal cystathionine□ synthase has anti-inflammatory properties (56) and may stimulate the production of mucins (57). Under these circumstances, colonization by AmII and AmIV strains may be pro-inflammatory if they decrease the effective concentration of H₂S at epithelial surfaces.

The relative competitive advantage of AmII and AmIV strains in antibiotic treated mice was unexpected given the relative high prevalence of AmI strains in human populations (16, 44). However, we noted that microbiota of SPF mice, which has an endogenous mouse AmI strain, provided colonization resistance which led us to ask if a similar phylogroup exclusion is observed in humans. In stool samples collected at various time points after various interventions aimed at reducing obesity, we observed dominance by single phylogroup. In some instances both 16S rRNA-based community profiling and qPCR indicated that A. muciniphila was not detectable in baseline samples yet appeared by 6 months (PID001 and PID009), and in others the dominant phylogroup disappeared within 3 month after baseline sample collection (PID033 and PID137), and yet in other patient samples a phylogroup would disappear and return (PID049) or be replaced by a new phylogroup (PID141, PID181 and PID182). The abrupt disappearance of Akkermansia has been previously documented in densely sampled individuals (58). At this, stage we cannot distinguish between population crashes that are followed by re-population by newly acquired Akkermansia phylogroup or blooms of pre-existing phylogroup that were present below the limits of detection. Evidence for the latter is supported by our ability to culture A. muciniphila strains that did not belong to the predominant phylogroup within the sample stool sample. Overall, patients are dominated by a single major phylogroup at any one time, but the abundance and identity of each A. muciniphila phylogroup is subject to fluctuation by environmental factors and ecological pressures still unknown.

The phenotypic diversity of the A. muciniphila strains in this cohort of patients suggests that experimental approaches using cultured strains will be critical to understand Akkermansia physiology. A recent survey of more than 10,000 adults in the American Gut Project, established a weak inverse correlation between A. muciniphila abundance and BMI, with a protective role against obesity when adjusted for confounders like sex, age and diet (11). It is plausible that these correlations may be further strengthened when stratified by what is the most prevalent A. muciniphila phylogroup in an individual. It is certainly possible that while some strains are beneficial, others may be neutral or even potentially harmful (59). Even strains that are considered beneficial, like MucT, may be harmful in the “wrong” context depending on the host's inflammatory status, diet or microbiota (60). The observation that patients can be colonized by different strains at different time suggests that A. muciniphila colonization is a dynamic process, especially considering that its primary food source, host mucins, should not be subject to the same variability as diet derived carbohydrates used by other intestinal microbes. Determining which A. muciniphila strains are most beneficial, and what factors influence strain-specific colonization, will be critical for the development of effective A. muciniphila based probiotics.

### METHODS

### Media, Strains and Growth Conditions

Bacteria were isolated and grown in an anaerobic chamber (Coy Laboratory) with the following gaseous characteristics: 5% hydrogen, 5% carbon dioxide, 90% nitrogen. A. muciniphila was grown in mucin medium based on previous work (7) (3 mM KH2PO4, 3 mM Na₂PO₄, 5.6 mM NH₄Cl, 1 mM MgCl₂, 1 mM Na₂S·9H₂O, 47 mM NaHCO₃, 1 mM CaCl₂ and 40 mM HCl, trace elements and vitamins (61), and 0.25% porcine gastric mucin (Type III, Sigma-Aldrich)). Additional media used to culture Akkermansia included synthetic media, where porcine gastric mucin was replaced with 0.2% GlcNAc, 0.2% glucose, 16 g/L of soy peptone and 4 g of threonine/L (14), and BD Bacto Brain Heart Infusion broth (BD #237500) with 0.25% porcine gastric mucin. To test growth with cysteine, mucin medium was supplemented with filter sterilized L-cysteine to a final concentration of 0.5 mM. The A. muciniphila strain Muc^{T} (7) was obtained from ATCC (BAA-835). HEK-Blue™ hTLR2/1/6 and hTLR4 were obtained from Invivogen (hkb-htlr2, hkb-htlr4) and maintained as described by the manufacturer. HT29-MTX was from Sigma (12040401-1VL) and maintained in DMEM (Gibco 11995-065) supplemented with 10% fetal bovine serum.

### Isolation of A. muciniphila from Fecal Samples

The recruitment criteria and composition of stool donors in the POMMS Study has been previously reported (18). Approximately 75 mg of frozen stool was used to inoculate 1 mL of mucin medium supplemented with vancomycin (6 □g/mL), gentamicin (10 □g/mL), and kanamycin (12 □g/mL), and incubated at 37° C. for 48 hours. After three sequential passages in mucin medium, a sample of the suspension was streaked on 1% agar mucin media plates to isolate single colonies and incubated for 7 days at 37° C. Colonies of unique morphology were restreaked on 1% agar BBL Brain Heart Infusion (BD Biosciences, 211065) plates supplemented with 0.2% mucin and incubated for 4 days. Total DNA was isolated and the strain identified by polymerase chain reaction (PCR)-based amplification of the 16S rRNA gene V3-V4 region. The nomenclature used for new A. muciniphila strains is as follows; AkkXXXYn, with X being patient number (009-275), Y the month of sample collection, (0,1.5,3,4.5, or 6) and n the clone typed if more that one colony was collected per plate (a-d; no letter indicates that only one colony was picked for analysis). AkkB40 is an isolate from a healthy adult male.

### Global analysis of Microbial Composition by 16S rRNA Sequencing

The composition of total bacteria in stool samples was determined from DNA samples extracted from stool with a Qiagen Stool extraction kit (Qiagen #51604) by amplification of the 16S rRNA gene by PCR using primers 515 and 806 as described in Earth Microbiome Project protocols, followed by DNA sequencing of resulting amplicons on an Illumina MiSeq platform. Phylogenetic analysis was performed using the Quantitative Insights into Microbial Ecology 2 (QIIME2) platform, version 2019.7 (62). Raw sequence data were demultiplexed using the emp-paired option (63, 64), followed by denoising with DADA2 (65) using parameters p-trim-left-f 10, p-trim-left-r 10, p-trunc-len-f 233, and p-trunc-len-r 164. Amplicon sequence variants (ASVs) were assigned taxonomy using the feature-classifier classify-sklearn (66, 67) and the SILVA 132 99% 515F/806R reference sequences (67).

### Phenotypic Characterization of A. muciniphila Isolates

Growth rates determination: Growth rates were assessed in both liquid mucin and synthetic medium. Starter cultures were grown to saturation in 3 mL synthetic medium supplemented with 0.25% porcine gastric mucin and diluted 1:5 into fresh medium and grown for an additional 8 h. The resulting cultures were then diluted 1:25 into fresh media (OD₆₀₀0.01-0.05) and 150 □l aliquots were dispensed into 96-well microplates. Each well was covered with 100 □l of paraffin oil and incubated at 37° C. in a BMG SpectroStar Nano plate reader under anaerobic conditions. The optical density (OD₆₀₀) was measured at 1 h intervals for 72 h. Generation times and growth rates were determined using the R package Growthcurver (version 0.3.0)(68). Results were obtained from three biological replicates per strain.

Growth with cysteine and sodium hydrogen sulfide: Growth was tested in medium supplemented with L-cysteine or the H₂S donor sodium hydrogen sulfide (NaSH) (Cayman Chemical, 10012555). NaSH stock solutions were prepared in PBS under anaerobic conditions. Akkermansia starter cultures were standardized to an OD₆₀₀ of 0.5 and diluted 1:25 into 3 ml of mucin media with or without 1 mM L-cysteine or 40 □M NaSH. The cultures were incubated anaerobically at 37° C. and the optical density was measured over 4 days. All assays were run in triplicate.

Agglutination: Actively growing cultures were used to innoculate 1.2 ml of mucin medium in fresh deep 96 well plates and incubated anaerobically at 37° C. for 3 days. The degree of bacterial sedimentation was quantified by removing 150 □L of culture from the top of the well Agglutination was calculated for each strain as:

$$agglutination = 1 - ( \frac{mean OD 600 of TOP}{mean OD 600 of WHOLE} )$$

Three biological replicates of this assay were performed for every strain, and each contain three technical replicates.

Adherence to epithelial cells: HT29-MTX cells were seeded into 96-well plates at a density of 2.5×10⁴ cells per well and grown for 7 days past confluence. Wells were washed twice with PBS and incubated with 2.5×10⁶ A. muciniphila cells in DMEM for 2 h at 37° C. under anaerobic conditions. As a control for non-specific binding of Akkermansia, UltraCruz high binding ELISA (sc-204463) plates were pre-coated with 100 □l of 1% Bovine Serum Albumin (BSA). Wells with HT29-MTX cells or coated with BSA were washed twice with PBS to remove non-adherent bacteria. Synthetic media (100 □l) was added to each well and plates were cultured for either 48 h or 96 h at 37° C. under anaerobic conditions. HT29-MTX cell or BSA binding was assessed by measuring the optical density at 600 nm after outgrowth in the assay wells and calculating the ratio of HT29-MTX coated OD:BSA coated OD. Data is reported as the average and standard deviation of 3 technical replicates from 3-4 independent biological replicates. For microscopy, HT29-MTX cells were seeded into 24-well plates with 12 mm round glass coverslips at a density of 1×10⁵ cells per well and grown for 7 d past confluence. Wells were washed twice with PBS and incubated with 1×10⁶ A. muciniphila cells in 500 ml anaerobic-adapted DMEM for 2 h at 37° C. under anaerobic conditions. Wells were washed twice, fixed with 3.7% formaldehyde in PBS for 30 min on ice, washed twice with PBS, and blocked overnight at 4° C. in blocking buffer (2%(w/v) BSA in PBS). Coverslips were incubated with a 1:50 dilution of anti-Akkermansia polyclonal antibody followed by an incubation with goat anti-rabbit-488 (Invitrogen A-11008), and Hoechst for 1 h at 25° C. After two washes, coverslips were mounted on slides with vectashield and imaged on Nikon Elipse Ti2 inverted microscope with 20×objective.

Measurement of short chain fatty acids (SCFA): For each strain, 1 mL of culture supernatants from strains grown in mucin medium was removed for SCFA analysis following the protocol of Holmes et al (69). In brief, the supernatant was centrifuged at 14,000 rcf for 5 min at 4° C. to pellet debris, then 750 uL of supernatant was passed through a 0.22 mm spin column filter. The resultant filtrate was then acidified to a pH<3 with 50 uL of 6N HCL, and transferred to a glass autosampler vial for analysis. Filtrates were analyzed on an Agilent 7890b gas chromatograph (GC) equipped with a flame-ionization detector (FID) and an Agilent HP-FFAP free fatty-acid column (69). The concentrations of acetate and propionate in the samples were determined using an 8-point standard curve (0.1 mM to 16 mM).

Sensitivity to ambient oxygen: Strains were grown from frozen stocks in 0.5 mL mucin (0.4%) medium in deep 96 well to saturation. After subculturing in mucin medium for 5 h, serial dilutions of each strain were spotted on BBL BHI agar (BD #211065) plates supplemented with mucin. One plate was left in the anaerobic chamber while the others were exposed to ambient O₂ for 12, 18 or 24 h, before returning to the chamber. The relative sensitivity to ambient oxygen was determined by monitoring the ratio of colony forming units (CFU) with and without exposure to ambient oxygen.

Bone marrow macrophage (BMW) stimulations: BMMs were obtained from 6-12-week-old C57BL/6J mice of the following genotypes: wild-type, Tlr2^{-/-}Tlr4^{-/-}Unc93b1^{3d/3d}, Tlr2^{-/-}Tlr4^{-/-}, Tlr2^{-/-}Unc93b1^{3d/3d}, and Tlr4^{-/-} (25). Bone marrow was dissociated through a 70-micron filter, treated with ACK Lysis Buffer (Gibco #:A1049201), and differentiated for 6 d in DMEM complete media (DMEM supplemented with 10% (v/v) fetal bovine serum, L-glutamine, penicillin-streptomycin, sodium pyruvate, HEPES, and 2-mercaptoethanol) supplemented with 10% (v/v) of supernatants from 3T3-CSF cells, overproducing macrophage colony stimulating factor. For stimulations, BMMs were plated in DMEM complete media supplemented with 10% (v/v) M-CSF and incubated with A. muciniphila at the indicated multiplicity of “infection” (MOI), 1 μM CpG-B (InvivoGen, tlr1-1668-1), 500 ng/mL Pam3CSK4 (InvivoGen, tlr1-pms), or 50 ng/mL LPS (InvivoGen, tlr1-3pelps). For analysis of secreted cytokines, the supernatant was collected 4 h after stimulation and analyzed with the BD Cytometric Bead Array Mouse Inflammation Kit (BD Biosciences, 552364) according to the manufacturer's instructions.

Activation of hTLRs: HEK-Blue™ hTLR2/1/6 and hTLR4 expressing cells (InvivoGen hkb-htlr2, hkb-htlr4) were seeded into 96 well plates pre-treated with poly-L-Lysine. A. muciniphila isolates were added to each well at an MOI of 5 in triplicate. Negative controls included culture media with 10% heat inactivated FBS. Positive controls included, ultrapure lipopolysaccharide from E. coli 055:B5 was used at 100 ng/mL and 1 ng/mL. For the experiments using hTLR2/1/6, Pam3CSK4, a synthetic triacylated lipopeptide, was used at concentrations of 100 ng/mL and 5 ng/mL. After a 16-hour incubation, levels of sAP were assessed with QUANTI-Blue™ detection media as detailed by the manufacturer.

### Genomic Sequencing, Annotation and Comparative Analysis

A. muciniphila genomic DNA was extracted using a MagAttract HMW DNA kit (Qiagen, 67563) according to the manufacturer's protocol. The extracted DNA was ethanol precipitated and the final concentration determined with a Qubit dsDNA HS kit (Thermo

Scientific). Libraries were generated using a SMRTbell Template Prep Kit 2.0 (Pacific Biosciences) and sequenced on a PacBio Sequel instrument.

After sequencing, PacBio SMRTLink software (version 8.0.0) was used to demultiplex the samples and the resulting BAM files were converted to fasta files using Samtools (70). Genome assembly was performed with Flye version 2.7 with the following parameters: -genome-size 3m-plasmids-meta (71). The assembled, circular genomes were then rotated to set the starting position to the dnaA gene using the fixstart function in Circulator (72). Finally, assembly annotation and quality evaluation were run using the PATRIC RASTtk-enabled Genome Annotation Service (73). Sequences have been deposited in Genbank (NCBI BioProject accession number PRJNA715455, incorporated by reference in their entireties).

Comparative analysis of the assembled genomes was run using the pangenomic workflow in Anvi'o version 6.2 (74, 75). First, the 43 assembly fasta files were reformatted into an Anvi'o compatible contig database by running the script anvi-script-FASTA-to-contigs-db. This command uses Prodigal to identify open reading frames (76). The resulting databases were annotated with the script anvi-run-ncbi-cogs, and subsequently combined to make a genome database using anvi-gen-genomes-storage. To compute the pangenome, the command anvi-pan-genome was run with the following parameters: -minbit 0.5, -mcl-inflation 10, -use-ncbi-blast.

The inventors computed average nucleotide identity (ANI) across the genomes using the command anvi-compute-genome-similarity with the —method pyani parameter (77). We included publicly available Akkermansia genomes as controls in our ANI and pangenome analyses. Representatives of phylogroups AmI, AmII, and AmIII were described in Guo et al. and retrieved from NCBI (17) (GenBank assembly accession: GCA_002885425.1, GCA_002885025.1, GCA_002884975.1, GCA_002884915.1, GCA_002885515.1, incorporated by reference in their entirety). The phylogroup AmIV representative genome CDI-150b was obtained from the JGI IMG database (16). Based on the resulting analysis, each genome was assigned to a phylogroup using the function anvi-import-misc-data, and phylogroup specific gene functions were then obtained using the command anvi-get-enriched-functions-per-pan-group to identify Cluster of Orthologous Groups of proteins (COGs). COGs present in all members of a given phylogroup, and absent in all other phylogroups, were considered to represent phylogroup specific gene functions. The adjusted q-value represents the false discovery rate adjusted p-value corrected for multiple testing as calculated by Anvi-o. Finally, the genomes were analyzed for metabolic pathways in Anvi'o (https://merenlab.org/software/anvio/help/main/programs/anvi-estimate-metabolism/). Each genome was annotated with the KEGG KOfam database using the program anvi-run-kegg-kofams (78, 79). The annotated genomes were then used as input to the program anvi-estimate-metabolism, with the flag-module-completion-threshold set to identify pathways with a minimum of 50% completion in at least one isolate genome. The modules output was further analyzed with the command anvi-compute-functional-enrichment to test for phylogroup specific enrichment. To visualize the data, heatmaps were generated using the pheatmap R package (80) and genes were plotted with the gggenes R package (81) in ggplot2 (82). To search for specific ASR genes among the isolates, custom BLAST databases were generated using the annotated isolate genomes (83). Searches were conducted using the sequences for the ASR genes from A. muciniphila Muc^{T} as the query.

To identify glycoside hydrolase families in the sequenced strains, we used dbCAN2 v2.0.11 to annotate carbohydrate-active enzymes (84). DNA fasta files were used as input and the annotation was run using the standalone tool run_dbcan. The resulting annotation tables were analyzed to determine the number of each type of glycoside hydrolase family per genome. Annotations detected by Diamond and at least one additional method, Hotpep or Hmmer, were considered positive.

### Mouse Colonization and Phylogroup Competitions

To prepare the inoculum for competition experiments, A. muciniphila cultures were standardized by optical density, combined using equal parts of each phylogroup to be tested and stored at −80° C. in PBS containing 20% glycerol.

All mouse experiments were approved by Duke's Institutional Animal Care and Use Committee. In vivo competition experiments were caried out in six-week-old female C57BL/6J mice obtained from Jackson Laboratories. A. muciniphila colonization was tested in mice both with and without pre-treatment with antibiotics (3 g/L tetracycline suspended in distilled water with 10% sucrose for two weeks). Following antibiotic treatment, clearance of residual mouse Akkermansia were determined by PCR using Akkermansia-specific 16S rRNA primers (12). For in vivo competition assays, mice were inoculated by intragastric gavage with a mixture containing 2.5×10⁸ CFU of each phylogroup in a total volume of 140 □l. The three-phylogroup competition experiment used a mixture of the strains Muc^{T} (AmIa), Akk1683 (AmIb), and Akk0580 (AmII). Two additional competition experiments were run, each using a combination of four clades. The first four-clade competition used a mixture of strains Muc^{T} (AmIa), Akk1570 (AmIb), Akk0580 (AmII), and Akk0490 (AmIV). The second four-clade competition used a mixture of strains Akk2670 (AmIa), Akk2650 (AmIb), Akk2680 (AmII), and Akk2750 (AmIV).

### Analysis of A. muciniphila Phylogroup Distribution in Fecal Samples from Mice and Humans

Phylogroup abundance was assessed by qPCR with phylogroup specific primers. PCR was run with PowerUp SYBR Master Mix reagent on a QuantStudio 3 real time PCR system (Applied Biosystems) using fast cycling mode. The abundance of Akkermansia was calculated as copy per gram fecal material. For human samples, the same DNA used for 16S rDNA sequencing was used as template for qPCR with phylogroup-specific primers (Table S4). Unless otherwise noted, statistical analysis and plots were generated with GraphPad Prism version 9.0.0.

### REFERENCES

1. Fan Y, Pedersen O. 2021. Gut microbiota in human metabolic health and disease. Nat Rev Microbiol 19:55-71.

2. Allaire J M, Crowley S M, Law H T, Chang S Y, Ko H J, Vallance B A. 2018. The Intestinal Epithelium: Central Coordinator of Mucosal Immunity. Trends Immunol 39:677-696.

3. Le Chatelier E, et al. 2013. Richness of human gut microbiome correlates with metabolic markers. Nature 500:541-546.

4. Sonnenburg E D, Sonnenburg J L. 2019. The ancestral and industrialized gut microbiota and implications for human health. Nat Rev Microbiol 17:383-390.

5. David L A, Maurice C F, Carmody R N, Gootenberg D B, Button J E, Wolfe B E, Ling A V, Devlin A S, Varma Y, Fischbach M A, Biddinger S B, Dutton R J, Turnbaugh P J. 2014. Diet rapidly and reproducibly alters the human gut microbiome. Nature 505:559-63.

6. Derrien M, Collado M C, Ben-Amor K, Salminen S, de Vos W M. 2008. The Mucin Degrader Akkermansia muciniphila is an Abundant Resident of the Human Intestinal Tract. Appl Environ Microbiol 74:1646-1648.

7. Derrien M, Vaughan E E, Plugge C M, de Vos W M. 2004. Akkermansia muciniphila gen. nov., sp. nov., a human intestinal mucin-degrading bacterium. Int J Syst Evol Microbiol 54:1469-1476.

8. Cani P D, de Vos W M. 2017. Next-Generation Beneficial Microbes: The Case of Akkermansia muciniphila. Front Microbiol 8:1-8.

9. Dao M C, et al. 2016. Akkermansia muciniphila and improved metabolic health during a dietary intervention in obesity: Relationship with gut microbiome richness and ecology. Gut 65:426-436.

10. Yassour M, Lim M Y, Yun H S, Tickle T L, Sung J, Song Y M, Lee K, Franzosa E A, Morgan X C, Gevers D, Lander E S, Xavier R J, Birren B W, Ko G P, Huttenhower C. 2016. Sub-clinical detection of gut microbial biomarkers of obesity and type 2 diabetes. Genome Med 8:1-14.

11. Zhou Q, Zhang Y, Wang X, Yang R, Zhu X, Zhang Y, Chen C, Yuan H, Yang Z, Sun L. 2020. Gut bacteria Akkermansia is associated with reduced risk of obesity: evidence from the American Gut Project. Nutr Metab (Lond) 17:90.

12. Collado M C, Derrien M, Isolauri E, De Vos W M, Salminen S. 2007. Intestinal integrity and Akkermansia muciniphila, a mucin-degrading member of the intestinal microbiota present in infants, adults, and the elderly. Appl Environ Microbiol 73:7767-7770.

13. Everard A, Belzer C, Geurts L, Ouwerkerk J P, Druart C, Bindels L B, Guiot Y, Derrien M, Muccioli G G, Delzenne N M, de Vos WM, Cani P D. 2013. Cross-talk between Akkermansia muciniphila and intestinal epithelium controls diet-induced obesity. Proc Natl Acad Sci 110:9066-9071.

14. Plovier H, Everard A, Druart C, Depommier C, Van Hul M, Geurts L, Chilloux J, Ottman N, Duparc T, Lichtenstein L, Myridakis A, Delzenne N M, Klievink J, Bhattacharjee A, van der Ark K C H, Aalvink S, Martinez L O, Dumas M-E, Maiter D, Loumaye A, Hermans M P, Thissen J-P, Belzer C, de Vos W M, Cani P D. 2017. A purified membrane protein from Akkermansia muciniphila or the pasteurized bacterium improves metabolism in obese and diabetic mice. Nat Med 23:107-113.

15. Depommier C, Everard A, Druart C, Plovier H, Van Hul M, Vieira-Silva S, Falony G, Raes J, Maiter D, Delzenne N M, de Barsy M, Loumaye A, Hermans M P, Thissen J-P P, de Vos W M, Cani P D. 2019. Supplementation with Akkermansia muciniphila in overweight and obese human volunteers: a proof-of-concept exploratory study. Nat Med 25:1096-1103.

16. Kirmiz N, Galindo K, Cross K L, Luna E, Rhoades N, Podar M, Flores G E. 2019. Comparative Genomics Guides Elucidation of Vitamin B 12 Biosynthesis in Novel Human-Associated Akkermansia Strains. Appl Environ Microbiol 86:e02117-19.

17. Guo X, Li S, Zhang J, Wu F, Li X, Wu D, Zhang M, Ou Z, Jie Z, Yan Q, Li P, Yi J, Peng Y. 2017. Genome sequencing of 39 Akkermansia muciniphila isolates reveals its population structure, genomic and functional diverisity, and global distribution in mammalian gut microbiotas. BMC Genomics 18:1-12.

18. McCann J R, Bihlmeyer N A, Roche K, Catherine C, Jawahar J, Kwee L C, Younge N E, Silverman J, Ilkayeva O, Sarria C, Zizzi A, Wootton J, Poppe L, Anderson P, Arlotto M, Wei Z, Granek J A, Valdivia R H, David L A, Dressman H K, Newgard C B, Shah S H, Seed P C, Rawls J F, Armstrong S C. 2021. The Pediatric Obesity Microbiome and Metabolism Study (POMMS): Methods, Baseline Data, and Early Insights. Obesity 29:569-578.

19. Xing J, Li X, Sun Y, Zhao J, Miao S, Xiong Q, Zhang Y, Zhang G. 2019. Comparative genomic and functional analysis of Akkermansia muciniphila and closely related species. Genes and Genomics 41:1253-1264.

20. Reunanen J, Kainulainen V, Huuskonen L, Ottman N, Belzer C, Huhtinen H, de Vos W M, Satokaria R. 2015. Akkermansia muciniphila adheres to enterocytes and strengthens the integrity of the epithelial cell layer. Appl Environ Microbiol 81:3655-3662.

21. Belzer C, Chia L W, Aalvink S, Chamlagain B, Piironen V, Knol J, de Vos W M. 2017. Microbial Metabolic Networks at the Mucus Layer Lead to Diet-Independent Butyrate and Vitamin B12 Production by Intestinal Symbionts. MBio 8:1-14.

22. Ouwerkerk J P, van der Ark K C H, Davids M, Claassens N J, Finestra T R, de Vos W M, Belzer C. 2016. Adaptation of Akkermansia muciniphila to the Oxic-Anoxic Interface of the Mucus Layer. Appl Environ Microbiol 82:6983-6993.

23. Ottman N, Reunanen J, Meijerink M, Pietilä T E, Kainulainen V, Klievink J, Huuskonen L, Aalvink S, Skurnik M, Boeren S, Satokari R, Mercenier A, Palva A, Smidt H, de Vos W M, Belzer C. 2017. Pili-like proteins of Akkermansia muciniphila modulate host immune responses and gut barrier function. PLoS One 12:e0173004.

24. Cario E, Gerken G, Podolsky D K. 2007. Toll-Like Receptor 2 Controls Mucosal Inflammation by Regulating Epithelial Barrier Function. Gastroenterology 132:1359-1374.

25. Sivick K E, Arpaia N, Reiner G L, Lee B L, Russell B R, Barton G M. 2014. Toll-like receptor-deficient mice reveal how innate immune signaling influences Salmonella virulence strategies. Cell Host Microbe 15:203-213.

26. Huh J W, Shibata T, Hwang M, Kwon E-H, Jang M S, Fukui R, Kanno A, Jung D J, Jang M H, Miyake K, Kim Y M. 2014. UNC93B1 is essential for the plasma membrane localization and signaling of Toll-like receptor 5. Proc Natl Acad Sci 111:7072-7077.

27. Majer O, Liu B, Woo B J, Kreuk L S M, Van Dis E, Barton G M. 2019. Release from UNC93B1 reinforces the compartmentalized activation of select TLRs. Nature 575:371-374.

28. Oliveira-Nascimento L, Massari P, Wetzler L M. 2012. The role of TLR2 ininfection and immunity. Front Immunol 3:1-17.

29. Pan Y J, Lin T L, Chen C T, Chen YY , Hsieh P F, Hsu C R, Wu M C, Wang J T. 2015. Genetic analysis of capsular polysaccharide synthesis gene clusters in 79 capsular types of Klebsiella spp. Sci Rep 5:15573.

30. Percy M G, Gründling A. 2014. Lipoteichoic Acid Synthesis and Function in Gram-Positive Bacteria. Annu Rev Microbiol 68:81-100.

31. May K L, Lehman K M, Mitchell A M, Grabowicz M. 2019. A stress response monitoring lipoprotein trafficking to the outer membrane. MBio 10:1-14.

32. Lau C K Y, Krewulak K D, Vogel H J. 2016. Bacterial ferrous iron transport: The Feo system. FEMS Microbiol Rev 40:273-298.

33. Achard M E S, Chen K W, Sweet M J, Watts R E, Schroder K, Schembri M A, McEwan A G. 2013. An antioxidant role for catecholate siderophores in Salmonella. Biochem J 454:543-549.

34. Peralta D R, Adler C, Corbalán N S, Paz García E C, Pomares M F, Vincent P A. 2016. Enterobactin as Part of the Oxidative Stress Response Repertoire. PLoS One 11:e0157799.

35. Kitamura M, Okuyama M, Tanzawa F, Mori H, Kitago Y, Watanabe N, Kimura A, Tanaka I, Yao M. 2008. Structural and functional analysis of a glycoside hydrolase family 97 enzyme from Bacteroides thetaiotaomicron. J Biol Chem 283:36328-36337.

36. Liu Q P, Yuan H, Bennett E P, Levery S B, Nudelman E, Spence J, Pietz G, Saunders K, White T, Olsson M L, Henrissat B, Sulzenbacher G, Clausen H. 2008. Identification of a GH₁₁₀ subfamily of α1,3-galactosidases: Novel enzymes for removal of the α3Gal xenotransplantation antigen. J Biol Chem 283:8545-8554.

37. Wu H, Rebello O, Crost E H, Owen C D, Walpole S, Bennati-Granier C, Ndeh D, Monaco S, Hicks T, Colvile A, Urbanowicz P A, Walsh M A, Angulo J, Spencer D I R, Juge N. 2020. Fucosidases from the human gut symbiont Ruminococcus gnavus. Cell Mol Life Sci 78:675-693.

38. Robbe C, Capon C, Maes E, Rousset M, Zweibaum A, Zanetta J P, Michalski J C. 2003. Evidence of regio-specific glycosylation in human intestinal mucins: Presence of an acidic gradient along the intestinal tract. J Biol Chem 278:46337-46348.

39. Makarova K S, Koonin E V. 2015. Annotation and Classification of CRISPR-Cas Systems, p. 47-75. In Lundgren, M, Charpentier, E, Fineran, P (eds.), CRISPR: Methods and Protocols. Humana Press, New York.

40. Chylinski K, Le Rhun A, Charpentier E. 2013. The tracrRNA and Cas9 families of type II CRISPR-Cas immunity systems. RNA Biol 10:726-737.

41. Sekowska A, Kung H F, Danchin A. 2000. Sulfur metabolism in Escherichia coli and related bacteria: Facts and fiction. J Mol Microbiol Biotechnol 2:145-177.

42. Makki K, Deehan E C, Walter J, Bäckhed F. 2018. The Impact of Dietary Fiber on Gut Microbiota in Host Health and Disease. Cell Host Microbe 23:705-715.

43. Guo X, Zhang J, Wu F, Zhang M, Yi M, Peng Y. 2016. Different subtype strains of Akkermansia muciniphila abundantly colonize in southern China. J Appl Microbiol 120:452-459.

44. Lv Q, Li S H, Zhang Y, Wang Y C, Peng Y, Zhang X X. 2020. A thousand metagenome-assembled genomes of Akkermansia reveal new phylogroups and geographical and functional variations in human gut. bioRxiv 2020.09.10.292292.

45. Jin M S, Kim S E, Heo J Y, Lee M E, Kim H M, Paik S G, Lee H, Lee J O. 2007. Crystal Structure of the TLR1-TLR2 Heterodimer Induced by Binding of a Tri-Acylated Lipopeptide. Cell 130:1071-1082.

46. Kang J Y, Nan X, Jin M S, Youn S J, Ryu Y H, Mah S, Han S H, Lee H, Paik S G, Lee J O. 2009. Recognition of Lipopeptide Patterns by Toll-like Receptor 2-Toll-like Receptor 6 Heterodimer. Immunity 31:873-884.

47. Bäckström M, Ambort D, Thomsson E, Johansson M E V., Hansson G C. 2013. Increased Understanding of the Biochemistry and Biosynthesis of MUC2 and Other Gel-Forming Mucins Through the Recombinant Expression of Their Protein Domains. Mol Biotechnol 54:250-256.

48. Lensmire J M, Hammer N D. 2019. Nutrient sulfur acquisition strategies employed by bacterial pathogens. Curr Opin Microbiol 47:52-58.

49. Tripp H J, Kitner J B, Schwalbach M S, Dacey J W H, Wilhelm L J, Giovannoni S J. 2008. SAR11 marine bacteria require exogenous reduced sulphur for growth. Nature 452:741-744.

50. Seif Y, Choudhary K S, Hefner Y, Anand A, Yang L, Palsson B O. 2020. Metabolic and genetic basis for auxotrophies in Gram-negative species. Proc Natl Acad Sci USA 117:6264-6273.

51. Carbonero F, Benefiel A C, Alizadeh-Ghamsari A H, Gaskins H R. 2012. Microbial pathways in colonic sulfur metabolism and links with health and disease. Front Physiol 3 November:1-11.

52. Ferrario C, Duranti S, Milani C, Mancabelli L, Lugli G A, Turroni F, Mangifesta M, Viappiani A, Ossiprandi M C, van Sinderen D, Ventura M. 2015. Exploring Amino Acid Auxotrophy in Bifidobacterium bifidum PRL2010. Front Microbiol 6:1-11.

53. Earley H, Lennon G, Balfe Á, Coffey J C, Winter D C, O'Connell PR. 2019. The abundance of Akkermansia muciniphila and its relationship with sulphated colonic mucins in health and ulcerative colitis. Sci Rep 9:15683.

54. Bian X, Wu W, Yang L, Lv L, Wang Q, Li Y, Ye J, Fang D, Wu J, Jiang X, Shi D, Li L. 2019. Administration of Akkermansia muciniphila Ameliorates Dextran Sulfate Sodium-Induced Ulcerative Colitis in Mice. Front Microbiol 10:1-18.

55. Png C W, Lindén S K, Gilshenan K S, Zoetendal E G, McSweeney C S, Sly L I, McGuckin M A, Florin T H J. 2010. Mucolytic bacteria with increased prevalence in IBD mucosa augment in vitro utilization of mucin by other bacteria. Am J Gastroenterol 105:2420-2428.

56. Wallace J L, Vong L, McKnight W, Dicay M, Martin G R. 2009. Endogenous and Exogenous Hydrogen Sulfide Promotes Resolution of Colitis in Rats. Gastroenterology 137:569-578.e1.

57. Motta J P, Flannigan K L, Agbor T A, Beatty J K, Blackler R W, Workentine M L, Da Silva G J, Wang R, Buret A G, Wallace J L. 2015. Hydrogen Sulfide Protects from Colitis and Restores Intestinal Microbiota Biofilm and Mucus Production. Inflamm Bowel Dis 21:1006-1017.

58. David L A, Materna A C, Friedman J, Campos-Baptista M I, Blackburn M C, Perrotta A, Erdman S E, Alm E J. 2014. Host lifestyle affects human microbiota on daily timescales. Genome Biol 15:R89.

59. Cirstea M, Radisavljevic N, Finlay B B. 2018. Good Bug, Bad Bug: Breaking through Microbial Stereotypes. Cell Host Microbe 23:10-13.

60. Seregin S S, Golovchenko N, Schaf B, Chen J, Pudlo N A, Mitchell J, Baxter N T, Zhao L, Schloss P D, Martens E C, Eaton KA, Chen G Y. 2017. NLRP6 Protects Il10−/− Mice from Colitis by Limiting Colonization of Akkermansia muciniphila. Cell Rep 19:733-745.

61. Stams A J M, Van Dijk J B, Dijkema C, Plugge C M. 1993. Growth of syntrophic propionate-oxidizing bacteria with fumarate in the absence of methanogenic bacteria. Appl Environ Microbiol 59:1114-1119.

62. Bolyen E, Rideout J R, Dillon M R, Bokulich N A, Abnet C C, Al-Ghalith G A, Alexander H, Alm E J, Arumugam M, Asnicar F, Bai Y, Bisanz J E, Bittinger K, Brejnrod A, Brislawn C J, Brown C T, Callahan B J, Caraballo-Rodríguez A M, Chase J, Cope E K, Da Silva R, Diener C, Dorrestein P C, Douglas G M, Durall D M, Duvallet C, Edwardson C F, Ernst M, Estaki M, Fouquier J, Gauglitz J M, Gibbons S M, Gibson D L, Gonzalez A, Gorlick K, Guo J, Hillmann B, Holmes S, Holste H, Huttenhower C, Huttley G A, Janssen S, Jarmusch A K, Jiang L, Kaehler B D, Kang K Bin, Keefe C R, Keim P, Kelley S T, Knights D, Koester I, Kosciolek T, Kreps J, Langille M G I, Lee J, Ley R, Liu Y X, Loftfield E, Lozupone C, Maher M, Marotz C, Martin B D, McDonald D, McIver L J, Melnik A V., Metcalf J L, Morgan S C, Morton J T, Naimey A T, Navas-Molina J A, Nothias L F, Orchanian S B, Pearson T, Peoples S L, Petras D, Preuss M L, Pruesse E, Rasmussen L B, Rivers A, Robeson M S, Rosenthal P, Segata N, Shaffer M, Shiffer A, Sinha R, Song S J, Spear J R, Swafford A D, Thompson L R, Tones P J, Trinh P, Tripathi A, Turnbaugh P J, Ul-Hasan S, van der Hooft J J J, Vargas F, Vázquez-Baeza Y, Vogtmann E, von Hippel M, Walters W, Wan Y, Wang M, Warren J, Weber K C, Williamson C H D, Willis A D, Xu Z Z, Zaneveld J R, Zhang Y, Zhu Q, Knight R, Caporaso J G. 2019. Reproducible, interactive, scalable and extensible microbiome data science using QIIME 2. Nat Biotechnol 37:852-857.

63. Hamady M, Knight R. 2009. Microbial community profiling for human microbiome projects: Tools, techniques, and challenges. Genome Res 19:1141-1152.

64. Hamady M, Walker J J, Harris J K, Gold N J, Knight R. 2008. Error-correcting barcoded primers for pyrosequencing hundreds of samples in multiplex. Nat Methods 5:235-237.

65. Callahan B J, McMurdie P J, Rosen M J, Han A W, Johnson A J A, Holmes S P. 2016. DADA2: High-resolution sample inference from Illumina amplicon data. Nat Methods 13:581-583.

66. Bokulich N A, Kaehler B D, Rideout J R, Dillon M, Bolyen E, Knight R, Huttley G A, Gregory Caporaso J. 2018. Optimizing taxonomic classification of marker-gene amplicon sequences with QIIME 2's q2-feature-classifier plugin. Microbiome 6:1-17.

67. Yilmaz P, Parfrey L W, Yarza P, Gerken J, Pruesse E, Quast C, Schweer T, Peplies J, Ludwig W, Glöckner F O. 2014. The SILVA and “All-species Living Tree Project (LTP)” taxonomic frameworks. Nucleic Acids Res 42:D643-D648.

68. Sprouffske K, Wagner A. 2016. Growthcurver: An R package for obtaining interpretable metrics from microbial growth curves. BMC Bioinformatics 17:17-20.

69. Holmes Z C, Silverman J D, Dressman H K, Wei Z, Dallow E P, Armstrong S C, Seed P C, Rawls J F, David L A. 2020. Short-chain fatty acid production by gut microbiota from children with obesity differs according to prebiotic choice and bacterial community composition. MBio 11:1-15.

70. Li H, Handsaker B, Wysoker A, Fennell T, Ruan J, Homer N, Marth G, Abecasis G, Durbin R. 2009. The Sequence Alignment/Map format and SAMtools. Bioinformatics 25:2078-2079.

71. Kolmogorov M, Yuan J, Lin Y, Pevzner P A. 2019. Assembly of long, error-prone reads using repeat graphs. Nat Biotechnol 37:540-546.

72. Hunt M, Silva N De, Otto T D, Parkhill J, Keane J A, Harris S R. 2015. Circlator: Automated circularization of genome assemblies using long sequencing reads. Genome Biol 16:1-10.

73. Brettin T, Davis J J, Disz T, Edwards R A, Gerdes S, Olsen G J, Olson R, Overbeek R, Parrello B, Pusch G D, Shukla M, Thomason J A, Stevens R, Vonstein V, Wattam A R, Xia F. 2015. RASTtk: A modular and extensible implementation of the RAST algorithm for building custom annotation pipelines and annotating batches of genomes. Sci Rep 5.

74. Eren AM, Esen O C, Quince C, Vineis J H, Morrison H G, Sogin M L, Delmont T O. 2015. Anvi'o: An advanced analysis and visualization platformfor 'omics data. PeerJ 2015:1-29.

75. Delmont T O, Eren E M. 2018. Linking pangenomes and metagenomes: The Prochlorococcus metapangenome. PeerJ 2018:1-23.

76. Hyatt D, Chen G L, LoCascio P F, Land M L, Larimer F W, Hauser L J. 2010. Prodigal: prokaryotic gene recognition and translation initiation site identification. BMC Bioinformatics 11:119.

77. Pritchard L, Glover R H, Humphris S, Elphinstone J G, Toth I K. 2016. Genomics and taxonomy in diagnostics for food security: soft-rotting enterobacterial plant pathogens. Anal Methods 8:12-24.

78. Aramaki T, Blanc-Mathieu R, Endo H, Ohkubo K, Kanehisa M, Goto S, Ogata H. 2020. KofamKOALA: KEGG Ortholog assignment based on profile HMM and adaptive score threshold. Bioinformatics 36:2251-2252.

79. Muto A, Kotera M, Tokimatsu T, Nakagawa Z, Goto S, Kanehisa M. 2013. Modular Architecture of Metabolic Pathways Revealed by Conserved Sequences of Reactions. J Chem Inf Model 53:613-622.

80. Kolde R. 2013. pheatmap: Pretty Heatmaps. R package version 1.0.12.

81. Wilkins D. 2020. gggenes: Draw Gene Arrow Maps in “ggplot2.” R package version 0.4.1.

82. Wickham H. 2009. ggplot2: elegant graphics for data analysis. Springer-Verlag, New York.

83. 2008. BLAST Command Line Applications User Manual [Internet]. National Center for Biotechnology Information, Bethesda (MD).

84. Zhang H, Yohe T, Huang L, Entwistle S, Wu P, Yang Z, Busk P K, Xu Y, Yin Y. 2018. DbCAN2: A meta server for automated carbohydrate-active enzyme annotation. Nucleic Acids Res 46:W95-W101.

1. A composition comprising at least one strain of Akkermansia muciniphila listed in Table S3 and a pharmaceutically acceptable carrier.

2. The composition of claim 1, wherein the composition comprises two or more strains of Akkermansia muciniphila listed in Table S3.

3. The composition of claim 2, wherein the two or more strains are from at least two different phylogroups.

4. The composition of claim 2, wherein the composition comprises three or more strains of Akkermansia muciniphila listed in Table S3.

5. The composition of claim 1, wherein the at least one strain of Akkermansia muciniphila is selected from the group consisting of Akk0090, Akk00915, Akk0093, Akk00945a, Akk0096, Akk01915, Akk0196, Akk0200, Akk0330, Akk0490a, Akk0496a, Akk0496b, Akk0500a, Akk0500b, Akk05415, Akk0580, Akk05815, Akk0880, Akk1370, Akk13715, Akk1376, Akk1410, Akk14115, Akk14745a, Akk14745b, Akk1476, Akk1570, Akk1573, Akk1576, Akk1610, Akk16115, Akk1613, Akk16145, Akk1616, Akk1683, Akk1700, Akk1713, Akk1750, Akk17515, Akk1756, Akk1813, Akk1820, Akk1826b, Akk1826d, Akk1863, Akk18645, Akk1866d, Akk1900, Akk1906, Akk1990, Akk2000, Akk2030, Akk2033, Akk2080, Akk2090, Akk21215, Akk2180, Akk2190, Akk2196, Akk2300, Akk2340, Akk2543, Akk2583, Akk2633, Akk2640, Akk2650, Akk2670, Akk2680, Akk2740, Akk2750, AkkB40, and combinations thereof.

6. The composition of claim 1, wherein the composition is formulated as a probiotic.

7. The composition of claim 1, wherein the composition is a capsule.

8. The composition of claim 1, wherein the at least one strain of Akkermansia muciniphila is live and pasteurized in the composition.

9. A method for the manufacture of a product, comprising:
a) providing a strain of Akkermansia muciniphila selected from Table S3; and
b) adding the strain to an edible composition, thereby obtaining the product.

10. A method of recolonizing the gut of a subject, the method comprising administering the composition of claim 1 to the subject in an amount capable of recolonizing the gut.

11. A method of altering the microbial composition of the gut of a subject, the method comprising administering the composition of claim 1 to the subject in an amount effective to alter the microbial composition within the subject.

12. A method of treating a disease or condition in a subject, the method comprising administering to the subject a therapeutically effective amount of the composition of claim 1 such that the disease or condition is treated.

13. The method according to claim 12, wherein the disease or condition is selected from the group consisting of cancer, metabolic disease, inflammatory conditions, neurological diseases, and combinations thereof

14. A method of reducing obesity in a subject in need thereof, the method comprising administering to the subject a therapeutically effective amount of the composition of claim 1 to reduce obesity.

15. The method of claim 12, wherein the disease is obesity, diabetes, or colitis.

16. A method of enhancing the response to cancer immunotherapies in a subject, the method comprising administering to the subject a therapeutically effective amount of the composition of claim 1.


SEQUENCE LISTING (16 sequences)

SEQ ID NO: 1 - DNA, 20 bp, Artificial Sequence
  Synthetic- Primer
cagcacgtgaaggtggggac

SEQ ID NO: 2 - DNA, 20 bp, Artificial Sequence
  Synthetic- Primer
ccttgcggttggcttcagat

SEQ ID NO: 3 - DNA, 20 bp, Artificial Sequence
  Synthetic- Primer
taattgcggcgagagaaacg

SEQ ID NO: 4 - DNA, 20 bp, Artificial Sequence
  Synthetic- Primer
acggccttggaaacttcaac

SEQ ID NO: 5 - DNA, 20 bp, Artificial Sequence
  Synthetic- Primer
ttttcctgacgaaccatgcc

SEQ ID NO: 6 - DNA, 20 bp, Artificial Sequence
  Synthetic- Primer
atgccgcacttttccacaag

SEQ ID NO: 7 - DNA, 20 bp, Artificial Sequence
  Synthetic- Primer
tgcgtgtaccacaattgctc

SEQ ID NO: 8 - DNA, 21 bp, Artificial Sequence
  Synthetic- Primer
tccgtgccgattcctatttcc

SEQ ID NO: 9 - DNA, 20 bp, Artificial Sequence
  Synthetic- Primer
tgctcagcagaaagaaccac

SEQ ID NO: 10 - DNA, 20 bp, Artificial Sequence
  Synthetic- Primer
tttaaagaaccgcgcgtgac

SEQ ID NO: 11 - DNA, 20 bp, Artificial Sequence
  Synthetic- Primer
tttgaaagcccgcatcatgc

SEQ ID NO: 12 - DNA, 20 bp, Artificial Sequence
  Synthetic- Primer
agcaccttattgacgatgcg

SEQ ID NO: 13 - DNA, 20 bp, Artificial Sequence
  Synthetic- Primer
tccaagcgaaagactgtgac

SEQ ID NO: 14 - DNA, 22 bp, Artificial Sequence
  Synthetic- Primer
cccagtatctccaatgttgctg

SEQ ID NO: 15 - DNA, 20 bp, Artificial Sequence
  Synthetic- Primer
tgattccgatacggacgctg

SEQ ID NO: 16 - DNA, 20 bp, Artificial Sequence
  Synthetic- Primer
ttgccatttccccggtactc